(19) United States
(12) Patent Application Publication
(10) Pub. No.: US 2012/0315248 A1
(43) Pub. Date: Dec. 13, 2012

(54) COMPOSITIONS AND METHODS FOR TREATING CANCER WITH ATTENUATED ONCOLYTIC VIRUSES

(75) Inventors: Anthony N. van den Pol, Branford, CT (US); Guido Wollmann, New Haven, CT (US)

(73) Assignee: Yale University

(21) Appl. No.: 13/464,692

(22) Filed: May 4, 2012

Related U.S. Application Data
(63) Continuation-in-part of application No. PCT/US2010/055480, filed on Nov. 4, 2010.
Application No. PCT/US2010/055480 is a continuation-in-part of application No. PCT/US2010/048472, filed on Sep. 10, 2010.
Application No. PCT/US2010/048472 is a continuation-in-part of application No. PCT/US2010/020370, filed on Jan. 7, 2010.
(60) Provisional application No. 61/257,962, filed on Nov. 4, 2009.

(51) Int. Cl. A61K 35/76; A61P 35/00

(52) U.S. Cl. 424/93.2

(57) ABSTRACT

Compositions including attenuated oncolytic viruses and methods of their use for the treatment of cancer are disclosed. Some attenuated virus exhibit potential as tumor therapies by exhibiting characteristics such as high selectivity, infectivity, cytotoxicity, or replication index for tumor cells, and/or low infectivity, cytotoxicity, or replication index for normal cells. In preferred embodiments, the ratio of replication of virus in normal cells:tumor cells is about 1:100 or greater. Preferred viruses have two or more mechanisms of attenuation including insertion of a transgene such as GFP or an interferon, preferably at position 1 of the viral genome. The compositions can be administered to subjects having tumors, in an effective amount to delay or inhibit the growth of a tumor, reduce the growth or size of the tumor, and/or inhibit or reduce metastasis of the tumor. Methods for manufacturing viruses and methods of testing their oncolytic potential are also disclosed.

### CROSS REFERENCE TO RELATED APPLICATIONS

This application is a continuation-in-part of PCT/US2010/055480, filed Nov. 4, 2010, which is a continuation-in-part of PCT/US2010/048472 filed Sep. 10, 2010, and a continuation-in-part of PCT/US2010/020370 filed Jan. 7, 2010, and claims priority to U.S. Provisional Patent Application No. 61/257,962 filed on Nov. 4, 2009.

### STATEMENT REGARDING FEDERALLY SPONSORED RESEARCH OR DEVELOPMENT

The United States government has certain rights in this invention by virtue of National Institutes of Health Grant Number CA124737 to Anthony N. van den Pol.

### FIELD OF THE INVENTION

The present application is generally related to compositions including attenuated oncolytic viruses and methods of their use for the treatment of cancer.

### BACKGROUND OF THE INVENTION

There is currently no cure for glioblastoma in the brain, and patients diagnosed with this type of cancer generally die within a year (Ohgaki, et al., J. Neuropathol. Exp. Neurol., 64:479-489 (2005)). Oncolytic viruses that can infect and destroy malignant glioblastomas have emerged as a potential approach to combating this cancer (Aghi, et al., Curr. Opinion, 7:419-430 (2000)). A potential complication in using oncolytic viruses to attack cancer is the problem of infection of normal cells. This is particularly problematic in the brain, where neurons do not replicate, and virally mediated neuronal loss could lead to unwanted dysfunction. An ideal oncolytic virus would show high levels of infection and replication in cancer cells but low levels in noncancer control cells.

In the field of oncolytic virus therapy, vesicular stomatitis virus (VSV) has emerged as a promising candidate. Preclinical studies have shown effectiveness against a variety of malignancies of the lung, colon (Stojdl, et al., Cancer Cell, 4:263-275 (2003)), liver (Shinozaki, et al., Hepatology, 41:196-203 (2005)), prostate (Ahmed, Virology, 330:34-49 (2004)), breast (Ebert, et al., Cancer Gene Ther., 12:350-358 (2005)), and white blood cells (Lichty, et al., Hum. Gene Ther., 15:821-831 (2004)). The oncolytic capabilities of VSV against brain tumors have previously been shown both in vitro and in vivo (Duntsch, et al., J. Neurosurg., 100; 1049-1059 (2004); Lun, et al., J. Natl. Cancer Inst., 98:1546-1557 (2006); Oezduman, et al., J. Neurosci., 28:1882-1893 (2008); van den Pol, et al., J. Comp. Neurol., 516:456-481 (2009)), but viral spread and neurovirulence in the brain remain challenging factors that need to be addressed in the consideration of VSV as a tool to target brain cancer.

As an oncolytic agent, VSV offers a number of advantages. Virus binding and internalization are facilitated through ubiquitous receptor mechanisms, allowing a large variety of different cancer types to be targeted (Stojdl, et al., Cancer Cell, 4:263-275 (2003)). This is particularly important for malignant brain tumors, which often display a histologically and genetically heterogeneous nature. VSV has been shown to target five different human brain tumor cell lines (Wollmann, et al., J. Virol., 79:6005-6022 (2005)), as well as primary glioblastoma cells derived from tissue from resective brain tumor surgery (Oezduman, et al., J. Neurosci., 28:1882-1893 (2008)). Another strong point of VSV oncolysis is a very fast lytic cycle, leading to fast tumor cell killing and release of new viral progeny in as little as 3 h; as the adaptive immune system mounts a defense against VSV, its rapid oncolytic action may enhance its ability to kill a brain tumor before the immune system eliminates the virus. In addition, systemic application has been shown to be effective in experimental models for targeting a variety of peripheral tumors (Ahmed, et al., Virology, 330:34-49 (2004); Shinozaki, et al., Hepatology, 41:196-203 (2005)), widespread metastatic tumors (Ebert, et al., Cancer Gene Ther., 12:350-358 (2005); Stojdl, et al., Cancer Cell, 4:263-275 (2003)), and brain tumors (Lun, et al., J. Natl. Cancer Inst., 98:1546-1557 (2006); Oezduman, et al., J. Neurosci., 28:1882-1893 (2008)). In summary, VSV has shown promise as an effective agent against malignant brain tumors. However, previous studies revealed the potential for infecting normal brain cells as one of the main challenges that need to be addressed before clinical trials can be pursued.

A number of recombinant VSVs that show attenuated virulence have been described. First, recombinant VSVs derived from DNA plasmids in general show weakened virulence (Rose, et al., Cell, 106:539-549 (2001)). Nucleotide changes that alter the amino acid composition in the M protein at position 51 result in attenuated VSV phenotypes in vitro (Coulon, et al., J. Gen. Virol., 71:991-996 (1990)) and in vivo (Ahmed, et al., Virology, 330:34-49 (2004); Clarke, et al., J. Virol., 81:2056-2064 (2007); Stojdl, et al., Cancer Cell, 4:263-275 (2003); Wu, et al., Hum. Gene Ther., 19:635-647 (2008)). The VSV transmembrane G protein is needed for binding and internalization; truncations in the G protein to generate a reduced number of cytoplasmic amino acids are also attenuated (Johnson, et al., Virology, 360:36-49 (2007); Schnell, et al., EMBO J., 17:1289-1296, (1989)). Altering the order of genes also attenuates the virus (Clarke, et al., J. Virol., 81:2056-2064 (2007); Cooper, et al., J. Virol., 82:207-219 (2008); Flanagan, et al., J. Virol., 75:6107-6114 (2001)). G gene deletions block the ability to produce infectious virus (Duntsch, et al., J. Neurosurg., 100:1049-1059 (2004)). Additionally, VSV-rp30, a wild-type-based VSV with an enhanced oncolytic profile, was developed through repetitive passage under evolutionary pressure (Wollmann, J. Virol., 79:6005-6022 (2005)).

However, there remains a need to identify viruses with enhanced selectivity and, or enhanced infectivity for tumor cells; reduced selectivity and, or reduced infectivity of normal, non-tumor cells; or preferably combinations thereof.

Therefore it is an object of the invention to provide pharmaceutical dosage units including attenuated viruses having at least two mechanisms of attenuation and having a viral replication ratio of at least 1:100 for normal cells compared to control cells.

It is a further object of the invention to provide pharmaceutical dosage units of attenuated viruses with improved selectivity for, and/or improved infectivity of, tumor cells compared to normal cells.

It is still a further object of the invention to provide pharmaceutical dosage units of attenuated viruses with decreased toxicity for normal cells.

It is still a further object of the invention to provide the pharmaceutical dosage unit at a higher dosage than possible for the wildtype or the parental strain.

It is another object of the invention to provide pharmaceutical dosage units of attenuated viruses in combination with a second therapeutic, for example interferon.

It is another object of the invention to provide methods for using pharmaceutical dosage units including high doses of attenuated viruses with improved selectivity for, improved infectivity of, and/or a higher index of replication in tumor cells compared to normal cells to treat cancer, particularly brain cancer.

It is still another object of the invention to provide methods for manufacturing attenuated viruses with improved selectivity for, improved infectivity of, and/or a higher index of replication in tumor cells compared to normal cells.

It is still a further object of the invention to provide methods for testing the activity of attenuated viruses.

### SUMMARY OF THE INVENTION

Compositions including attenuated oncolytic viruses and methods of their use for the treatment of cancer are disclosed. Some attenuated virus exhibit potential as tumor therapies by exhibiting characteristics including, but not limited to, high selectivity, infectivity, cytotoxicity, or replication index for tumor cells, and/or low infectivity, cytotoxicity, or replication index for normal cells.

One important index of oncolytic potential is the ratio of viral replication in normal/control cells versus tumor or cancer cells. These ratios serve as an important index of the relative levels of viral replication in normal and tumor cells. A large ratio indicates greater replication in cancer cells than in control cells. In preferred embodiments, the ratio of replication of virus in normal cells:tumor cells is about 1:100 or greater.

Preferred viruses have two or more mechanisms of attenuation. Mechanisms of attenuation include, but are not limited to, expression of the virus as a recombinant virus from vector DNA, G protein truncations and whole gene deletions, amino acid mutations and deletions of the M protein, spontaneous mutations induced by evolutionary pressure, and insertion of a transgene, preferably at position 1 of the viral genome. Some of the disclosed viruses contain a fluorescent reporter gene, for example GFP or preferably RFP at position one of the viral genome. The RFP (dsRed) combines to form a red tetramer, and this tetramer may have slightly greater toxicity than GFP. It is believed this reduces replication and budding of progeny VSV-p1-RFP and increases the toxicity of the virus when a cancer cell is infected.

Viruses may be modified to express one or more targeting or therapeutic proteins, separately or as a part of other expressed proteins. VSV has a good oncolytic profile, in-part, by taking advantage of defects in the innate cellular anti-viral defense system, which is a common feature in malignancies, including colon, breast, prostate, liver, and leukemia. Reduction in interferon-related antiviral defenses enhances infection of cancer cells by attenuated VSV viruses. In some embodiments the attenuated virus is engineered to express a therapeutic protein, such as an interferon, that provides an increase in protection against the virus to normal cells, but little or no protection to tumor cells.

The disclosed attenuated oncolytic viruses can be used to treat patients with tumors including cancer. The compositions can be administered to subjects, preferably mammals, most preferably humans, having benign or malignant tumors, in an effective amount to delay or inhibit the growth of a tumor in a subject, reduce the growth or size of the tumor, inhibit or reduce metastasis of the tumor, and/or inhibit or reduce symptoms associated with tumor development or growth. The types of tumors that can be treated with the provided compositions and methods include, but are not limited to, vascular tumors such as multiple myeloma, adenocarcinomas and sarcomas, tumors of bone, bladder, brain, breast, cervical, colo-rectal, esophageal, kidney, liver, lung, nasopharangeal, pancreatic, prostate, skin, stomach, and uterine. In the most preferred embodiments, an attenuated, oncolytic VSV is used to treat a brain tumor, preferably glioblastoma.

The particular mode of administration selected will depend upon factors such as the particular formulation, the severity of the state of the subject being treated, and the dosage required to induce an effective response. In a preferred embodiment the compositions are formulated for systemic or local delivery by injection. In alternate embodiments, the compositions are formulated for mucosal administration, such as through pulmonary, buccal, or most preferably nasal delivery. Actual dosage, or viral titer will depend on the oncolytic activity of the virus. For attenuated viruses exhibiting lower cytotoxicity for normal cells, a patient may be able to tolerate a high viral titer. For attenuated viruses exhibiting increased cytotoxicity for target cells, such as tumor or cancer cells, it may be desirable to administer a low viral titer. The most desirable virus will have high specific activity for tumor cells, and low cytotoxicity toward normal cells.

Administration of the disclosed compositions can be coupled with surgical, radiologic, other therapeutic approaches to treatment of cancer. For example, oncolytic viruses can be co-administered with chemotherapeutic agents, or therapeutic proteins such as an interferon. In some cases, cancer cells are resistant to infection by viruses such as VSV. For example, synovial sarcoma is shown to be highly resistant to infection by VSV, human cytomegalovirus, and Sindbis virus due to heightened basal expression of interferon-stimulated genes. For these viruses, an interferon inhibitor, such as valporoate, Jak1 inhibitor, or vaccinia virus B18R protein may be used to enhance susceptibility of the cancer to treatment with attenuated, oncolytic VSV.

Methods for manufacturing viruses and methods of testing their oncolytic potential, including infectivity, cytotoxicity, replication index, target cell specificity, and cell viability are also disclosed.

### BRIEF DESCRIPTION OF THE DRAWINGS

FIG. 1 is a diagram mapping the genomes of ten (10) attenuated VSV viruses compared to wildtype VSV. From top to bottom the viruses are 1) wildtype VSV showing the relative locations of the genomic regions encoding the N, P, M, G, and L proteins; 2) VSV-G/GFP showing the addition of a second copy of the G gene and GFP inserted between the native G and L protein encoding regions; 3) VSV-dG-GFP showing the addition of GFP in the first position, and deletion of the complete G protein encoding region; 4) VSV-dR-GFP showing the addition of RFP in the first position, and deletion of the complete G protein encoding region; 5) VSV-CT9 showing a truncation of the G protein and the addition of GFP between the G and L protein encoding regions; 6) VSV-CT1 showing a truncation of the G protein; 7) VSV-M51 showing deletion of the amino acid at position 51 of the M protein, and the addition of GFP between the G and L protein encoding regions; 8) VSV-CT9-M51 showing a truncation of the G protein, deletion of the amino acid at position 51 of the M protein, and the addition of GFP between the G and L protein encoding regions; 9) VSV-1p-GFP showing the addition of GFP in the first position; 10) VSV-1p-RFP showing the addition of RFP in the first position; 11) VSV-rp30 showing amino acid substitution mutations in the regions encoding the P and L proteins.

FIG. 2A is two bar graphs showing viability (% of control), at thirty-six hours post-infection, of normal, human glia cells infected with mock (control), or 0.5 MOI of one of ten (10) attenuated viruses with (right hand graph) or without (left hand graph) IFN-α treatment. FIG. 2B is two bar graphs showing viability (% of control), at seventy-two hours post-infection, of normal, human glia cells infected with mock (control), or 0.5 MOI of one of ten (10) attenuated viruses with (right hand graph) or without (left hand graph) IFN-α treatment. FIG. 2C is two bar graphs showing viability (% of control), at thirty-six hours post-infection, of U87 human glioblastoma cells infected with mock (control), or 0.5 MOI of one of ten (10) attenuated viruses with (right hand graph) or without (left hand graph) IFN-α treatment. FIG. 2D is two bar graphs showing viability (% of control), at seventy-two hours post-infection, of U87 human glioblastoma cells infected with mock (control), or 0.5 MOI of one of ten (10) attenuated viruses with (right hand graph) or without (left hand graph) IFN-α treatment.

FIG. 3A-3J are a series of line graphs showing viral titer (log₁₀ pfu/ml) over time (days post infection (d.p.i.)) normal, human glia cells infected with 1.0 MOI of one of ten (10) attenuated viruses with (-▴-) or without (-Δ-) IFN-α treatment.

FIG. 4A-4J are a series of line graphs showing viral titer (log₁₀ pfu/ml) over time (days post infection (d.p.i.)) U87 human glioblastoma cells infected with 1.0 MOI of one of ten (10) attenuated viruses with (-▴-) or without (-Δ-) IFN-α treatment. Graphs for replication-restricted VSV-dG variants display the baseline for the original inoculum.

FIG. 5A is a bar showing cell growth suppression (cell number as a percent (%)) of U-118, U-373, or A-172 human glioblastoma cells infected with 2.0 MOI of rp30, M51, CT9-M51, or p1-GFP attenuated VSV viruses. FIG. 5B is a bar graph showing GFP expression (GFP-positive cells as a percent (%) of total cells) of U-118, U-373, or A-172 human glioblastoma cells infected with 2.0 MOI of rp30, M51, CT9-M51, or p1-GFP attenuated VSV viruses. FIG. 5C is a bar graph showing MxA gene expression (fold induction normalized to VSV-G/GFP) of U-118, U-373, or A-172 human glioblastoma cells infected with VSV-G/GFP, VSV-rp30, VSV-p1-GFP, VSV-M51, or VSV-CT9-M51 attenuated VSV viruses, or control. Results are means for triplicate cultures. Error bars indicate standard errors of the means.

FIG. 6A is a line graph showing the percent (%) survival over time (days post infection (d.p.i.)) of sixteen day-old mice following intranasal injection with 500,000 plaque forming units (PFU) of VSV-G/GFP (solid line, n=10), or VSV-1p-GFP (broken line, n=10). FIG. 6B is a line graph showing the percent (%) body weight over time (days post infection (d.p.i.)) of sixteen day-old mice following intranasal injection with 500,000 plaque forming units (PFU) of VSV-G/GFP (--, n=10, 8 of which died during the assay as shown in FIG. 6A), or VSV-1p-GFP (-□-, n=10). n=10, the number of mice initially infected with the virus.

FIGS. 7A-7B are a series of bar graphs showing infectivity (%) of thirteen human sarcoma lines infected at 5 PFU/cell with either VSV-G/GFP (A) or VSV-rp30a (B) assessed at 12 hours post-infection (hpi) (white bars) and at 36 hpi (black bars). FIG. 7C is a bar graph showing killing (%) of the human sarcoma lines mock infected (white bars) or infected at 5 PFU/cell with VSV-G/GFP (gray bars) or VSV-rp30a (black bars). At 36 hpi, cells were incubated with ethidium homodimer (EtHD-1) and the percentage of cells fluorescing red was assessed. FIG. 7D is a bar graph showing replication (PFU/ml (×10⁷) (FIG. 7D).

FIGS. 8A to 8B are bar graphs showing percentage of normal human fibroblasts (hFib, bar 1), normal human decidua (hDecid, bar 2), mouse brain vascular endothelium (mBVE, bar 3), malignant peripheral nerve sheath tumor (MPNS, bar 4), Ewing's sarcoma family tumor (ESFT, bar 5), or osteosarcoma (Osteo, bar 6) expressing GFP after infection with VSV-rp30a at MOI 0.2 PFU/cell, without (A) or with (B) IFN pretreatment (100 U/ml for 6 h).

FIG. 9 is a graph showing tumor size (relative to day 0) in SCID mice with subcutaneous (s.c.) A673 xenografts that were left uninfected (dotted line) or injected intravenously (i.v.) with a single dose of 5.0E7 PFU of VSV-rp30a (solid line) on day zero. Data points represent the mean volume, relative to day zero, for tumors under each condition. Error bars, SEM.

FIG. 10A is a bar graph showing cell-associated genomes in virus-resistant SW982 (white bars) and virus susceptible S462-TY (black bars) that were incubated with 10 PFU/cell of virus 20 min at 4° C. (bar 1), 30 min at 37° C. (bar 2), 90 min at 37° C. (bar 3), or 90 min at 37° C. in the presence of 5 mM ammonium chloride (bar 4). After washing, cell-associated genomes from quadruplicate wells were assessed by quantitative RT-PCR normalized to β-actin. Results are normalized to SW982. Error bars, SEM. FIGS. 10B and 10C are bar graphs showing the percentage of synovial sarcoma SW982 (FIG. 10B) or control sarcoma SJSA-1 (FIG. 10C) cells positive for GFP after infection in triplicate by VSV-G/GFP or VSV-rp30a at MOI 0.5, 5, or 50 PFU/cell assessed 12 hpi (gray bars) and 36 hpi (black bars). Error bars, SEM.

FIGS. 11A to 11C are bar graphs showing baseline MxA (FIG. 11A), ISG-15 (FIG. 11B), and IFN-13 (FIG. 11C) mRNA expression (relative to fibroblasts) in uninfected cultures of human fibroblasts (hFibro, bar 1), VSV resistant synovial sarcoma SW982 (bar 2), and three VSV-susceptible sarcomas (ESFT A673 (bar 3), Osteosarcoma STSA1 (bar 4), and MPNS tumor S562-TY (bar 5)) measured from triplicate samples by quantitative RT-PCR and normalized to β-actin. Error bars, SEM.

FIG. 12A is a bar graph showing MxA (bar I), OAS-1 (bar 2), ISG-56 (bar 3), and ISG-15 (bar 4) mRNA levels in SW-S (column 1), SW-R (column 2), SW-R pretreated for 14 h with VPA (column 3), or SW-R pretreated with vaccinia B18R for 4 days (column 4). Triplicate samples were analyzed. Error bars, SEM. FIG. 12 B is a bar graph showing MxA mRNA (fold stimulation) in triplicate human glial cultures exposed to fresh medium (bar 1), fresh medium with 200 U/ml of IFN (bar 2), or medium conditioned for 1 day (bar 3), 2 days (bar 4), or 3 days (bar 5) on SW-R. After 6 h of exposure, glial cell RNA was harvested and analyzed for MxA mRNA. Error bars, SEM.

FIG. 13 is a bar graph showing IFN-β mRNA expression (fold increase) 10 hours after viral infection of SW-S, SW-R, or SW-R+B18R (4 days) with VSV-rp30a assessed by qRT-PCR from parallel triplicate cultures. Measurements were normalized to β-actin. Error bars, SEM.

FIG. 14 is a bar graph showing percentage of SW-R (column 1), liposarcoma SW872 (column 2), and bladder transitional cell carcinoma T24 (column 3) that were infected 20 hours after the cells were infected with VSV-rp30a at an MOI of 1 PFU/cell and exposed to 500 nM Jak-1 inhibitor either at the time of infection with VSV-rp30a (cotreatment, bar 2), 4 days in advance of infection (pretreatment, bar 3), or not at all (untreated, bar 1). Triplicate wells were analyzed. Error bars, SEM.

### DETAILED DESCRIPTION OF THE INVENTION

### I. Definitions

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. With respect to nucleic acids, the term “isolated” includes any non-naturally-occurring nucleic acid sequence, since such non-naturally-occurring sequences are not found in nature and do not have immediately contiguous sequences in a naturally-occurring genome.

As used herein, a “variant,” “mutant,” or “mutated” polynucleotide contains at least one polynucleotide sequence alteration as compared to the polynucleotide sequence of the corresponding wild-type or parent polynucleotide. A “variant,” “mutant,” or “mutated” polypeptide contains at least one amino acid sequence alteration as compared to the amino acid sequence of the corresponding wild-type, or parent polypeptide. Mutations may be natural, deliberate, or accidental.

As used herein, the term “nucleic acid(s)” refers to any nucleic acid containing molecule, including, but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. In accordance with standard nomenclature, nucleic acid sequences are denominated by either a three letter, or single letter code as indicated as follows: adenine (Ade, A), thymine (Thy, T), guanine (Gua, G) cytosine (Cyt, C), uracil (Ura, U).

As used herein, the term “polynucleotide” refers to a chain of nucleotides of any length, regardless of modification (e.g., methylation).

The term “gene” refers to a nucleic acid (e.g., DNA or RNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid molecule encoding,” refers to the order or sequence of nucleotides along a strand of nucleotides. The order of these nucleotides determines the order of amino acids along the polypeptide (protein) chain. The nucleotide sequence thus codes for the amino acid sequence

As used herein, the term “polypeptide” refers to a chain of amino acids of any length, regardless of modification (e.g., phosphorylation or glycosylation). In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, a “variant,” “mutant,” or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, a “nucleic acid sequence alteration” can be, for example, a substitution, a deletion, or an insertion of one or more nucleotides. An “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

As used herein, the terms “neoplastic cells,” “neoplasia,” “tumor,” “tumor cells,” “cancer” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign.

As used herein, “attenuated” refers to procedures that weaken an agent of disease (a pathogen). An attenuated virus is a weakened, less vigorous virus. A vaccine against a viral disease can be made from an attenuated, less virulent strain of the virus, a virus capable of stimulating an immune response and creating immunity but not causing illness or less severe illness. Attenuation can be achieved by chemical treatment of the pathogen, through radiation, or by genetic modification, using methods known to those skilled in the art. Attenuation may result in decreased proliferation, attachment to host cells, or decreased production or strength of toxins. Viruses may be attenuated for normal cells, tumor cells, or both.

As used herein “higher/greater/improved/increased oncolytic potential,” or “higher/greater/improved/increased oncolytic activity” of a virus includes, but is not limited to an increase in specificity, infectivity, index of replication, or other criteria of toxicity of a virus in a cell of interest, such as a tumor cell, compared to a normal or control cell; or a decrease in the infectivity, index of replication, or other criteria of toxicity in normal cells of one virus relative to another virus, or under a change in conditions such as the addition of a second therapeutic agent. In some comparisons, the first virus is a wildtype or parental strain, and the second virus is a variant, mutant, or attenuated virus. In some comparisons the two viruses are unrelated.

As used herein “lower/less/reduced/decreased oncolytic potential,” or “lower/less/reduced/decreased oncolytic activity” of a virus includes, but is not limited to a decrease in the specificity, infectivity, index of replication, or other criteria of toxicity of a cell of interest, such as a tumor cell, compared to a normal or control cell; or an increase in infectivity, index of replication, or other criteria of toxicity of a virus in normal cells; or of one virus relative to another virus, or under a change in conditions such as the addition of a second therapeutic agent. In some comparisons, the first virus is a wildtype or parental strain, and the second virus is a variant, mutant, or attenuated virus. In some comparisons the two viruses are unrelated.

### II. Compositions

The viruses disclosed herein may be “native” or naturally-occurring viruses, or engineered viruses, such as recombinant viruses. Mutations and other changes can be introduced into the viral genome to provide viruses with enhanced selectivity and cytolytic activity for cells of interest, such as cancer cells. In the most preferred embodiments, the virus is a Vesicular stomatitis virus (VSV).

VSV, a member of the Rhabdoviridae family, is enveloped and has a negative-strand 11.2-kb RNA genome that comprises five protein-encoding genes (N, P, M, G, and L) (Lyles, et al., Fields virology, 5^{th} ed., Lippincott Williams & Wilkins, 1363-1408 (2007)). It is a nonhuman pathogen which can cause mild disease in livestock. Infection in humans is rare and usually asymptomatic, with sporadic cases of mild flu-like symptoms. VSV has a short replication cycle, which starts with attachment of the viral glycoprotein spikes (G) to an unknown but ubiquitous cell membrane receptor. Nonspecific electrostatic interactions have also been proposed to facilitate viral binding (Lyles, et al., Fields virology, 5^{th} ed., Lippincott Williams & Wilkins, 1363-1408 (2007)). Upon internalization by clathrin-dependent endocytosis, the virus-containing endosome acidifies, triggering fusion of the viral membrane with the endosomal membrane. This leads to release of the viral nucleocapsid (N) and viral RNA polymerase complex (P and L) into the cytosol.

The viral polymerase initiates gene transcription at the 3′ end of the nonsegmented genome, starting with expression of the first VSV gene (N). This is followed by sequential gene transcription, creating a gradient, with upstream genes expressed more strongly than downstream genes. Newly produced VSV glycoproteins are incorporated into the cellular membrane with a large extracellular domain, a 20-amino-acid transmembrane domain, and a cytoplasmic tail consisting of 29 amino acids. Trimers of G protein accumulate in plasma membrane microdomains, several of which congregate to form viral budding sites at the membrane (Lyles, et al., Fields virology, 5^{th} ed., Lippincott Williams & Wilkins, 1363-1408 (2007)). Most cells activate antiviral defense cascades upon viral entry, transcription, and replication, which in turn are counteracted by VSV matrix protein (M). VSV M protein's multitude of functions include virus assembly by linking the nucleocapsid with the envelope membrane, induction of cytopathic effects and apoptosis, inhibition of cellular gene transcription, and blocking of host cell nucleocytoplasmic RNA transfer, which includes blocking of antiviral cellular responses (Ahmed, et al., Virology, 237:378-388 (1997)).

A. Oncolytic Viruses

Suitable VSV strains or serotypes that may be used include VSV Indiana, VSV New Jersey, VSV Chandipura, VSV Isfahan, VSV San Juan, and VSV Glasgow. Viruses can be naturally occurring viruses, or strains modified, for example, to increase or decrease the virulence of the virus, and/or increase oncolytic potential, increase the specificity or infectivity or index of replication of the virus particularly for tumor cells, and/or decrease the toxicity for normal cells compared to the parental strain. A number of VSV variants have been described. See for example (Clarke, et al., J. Virol., 81:2056-64 (2007), Flanagan, et al., J. Virol., 77:5740-5748 (2003), Johnson, et al., Virology, 360:36-49 (2007), Simon, et al., J. Virol., 81:2078-82 (2007), Stojdl, et al., Cancer Cell, 4:263-275 (2003)), WO 10/080,909, U.S. Published Application No. 2007/0218078, and U.S. Published Application No 2009/0175906.

B. Mechanisms of Attenuation

1. Recombinant VSVs

Recombinant VSVs derived from DNA plasmids in general show weakened virulence (Rose, et al., Cell, 106:539-549 (2001)). Attenuation of VSV phenotype can also be accomplished by one or more nucleotide sequence alterations that result in substitution, deletion, or insertion of one or more amino acids of the polypeptide it encodes.

2. G Protein Mutants

It may be desirable to attenuate virus growth, and/or block the ability to produce infectious virus, (Duntsch, et al., J. Neurosurg., 100:1049-1059 (2004)), for example, by deleting or mutating the viral glycoprotein (G protein). The VSV transmembrane G protein is needed for binding and internalization, and truncations in the G protein to generate a reduced number of the 29 cytoplasmic amino acids result in attenuated virus (Johnson, et al., Virology, 360:36-49 (2007), Schnell, et al., EMBO J., 17:1289-1296 (1998)). In some embodiments 1, 2, 3, 4, 5, or more amino acids are deleted from the G protein. For example the cytoplasmic portion of the G protein can be truncated from 29 amino acids to nine amino acids (VSV-CT9) or a single amino acid (VSV-CT1). VSV-CT1 and VSV-CT9 were made in Jack Rose lab for use in immunization, as described by Schnell, et al., EMBO J, 17:1289-1296 (1998). PMID: 9482726.

Although the CT1 mutant may show an attenuated phenotype in vivo (Johnson, et al., Virology, 360:36-49 (2007); Publicover, et al., J. Virol., 78:9317-9324 (2004)), as shown in the Examples below, low titers of this virus were not effective at killing glioblastoma cells. The VSV-CT9 mutant, with a G protein cytoplasmic domain truncated down to 9 amino acids, was only mildly impaired in viral budding but showed a greater degree of infection of glioblastomas than did the VSV-CT1 mutant. However, viruses having M protein mutations and those with insertion of transgene or report gene in the first position were more effective as attenuated oncolyic viruses.

As shown in the Examples, complete deletion of the glycoprotein G gene (for example in the VSV-dG-GFP and VSV-dG-RFP viruses described below) also attenuates the virus. In the absence of VSV glycoprotein, viral budding is severely impaired, with viral particle yields around 30 times lower than those with the G protein present (Schnell, et al., Cell, 90:849-857 (1997)). Though virus progeny can still be produced and leave the cell (Schnell, EMBO J., 17:1289-1296 (1998); van den Poi, J. Comp. Neural., 516:456-481 (2009)), the absence of G protein spikes leaves the viral particle incapable of binding to any new cell, thereby terminating the viral infectious cycle. This virus is effective at killing the cells it infects, but as its progeny are not infective. It is believed that it would be deployed most effectively as a direct tumor toxin (Duntsch, et al., J. Neurosurg., 100:1049-1059 (2004)). Use of a G protein deletion virus may require the addition of exogenous G protein, or expression of the G protein in trans, as described in the examples below, to prepare a composition containing virus that can effectively infect cells. By generating the virus in cells that express the VSV-G protein (Publicover, et al., J. Virol., 79:13231-13238 (2005)), the replication-restricted viruses, such G protein deletion strains, will undergo at least a single round of infection.

While increasing its safety profile in the brain, G protein deletion viruses will ultimately eliminate only those cancer cells directly infected upon direct inoculation into the tumor. Therefore, viruses characterized by deletion of entire G protein encoding region, such as VSV-dG-GFP, are particularly useful for a transient treatment delivered directly to the tumor site. These viruses may be useful, for example, to reduce tumor burden prior to surgery. Furthermore, clinical tumor-specific administration of the virus is believed to generate an immune response which may be effective in stimulating an antitumor immune response. It is believed that VSV can enhance destruction of tumors both by direct oncolytic actions and by recruiting the immune system to attack tumor cells (Qiao, et al., Gene Ther., 15:604-616 (2008)).

3. M Protein Mutants

Another strategy is to attenuate viral pathogenicity by reducing the ability of the virus to suppress host innate immune responses without compromising the yield of infectious progeny. This can be accomplished by mutating the M protein as described, for example, in Ahmed, J. Virol., 82(18):9273-9277 (2008). The M protein is a multifunctional protein that is involved in the shutoff of host transcription, nuclear cytoplasmic transport, and translation during virus infection (Lyles, Microbial. Mol. Biol. Rev. 64:709-724 (2000)). Mutation and/or deletion of one or more amino acids from the M protein, for example, MΔ51, or M51A can result in viral protein that is defective at inhibiting host gene expression. These mutations impair the virus's capability to shut down host cell gene expression while remaining functional for virus assembly (Coulon, et al., J. Gen. Virol., 71:991-996 (1990)). Effective mutations at position 51 of the matrix protein, by amino acid substitution (arginine for methionine) (Coulon, et al., J. Gen. Virol., 71:991996 (1990)) or methionine deletion (Publicover, et al., J. Virol., 80:7028-7036 (2006), Stojdl, et al., Cancer Cell, 4:263-275 (2003)), prevent the normal ability of VSV to block nuclear pores and thereby block cellular mRNA transport through the nuclear membrane. Without inhibition of gene expression, cells infected by VSV-M51 mutants mount a significantly greater interferon response, hence creating a stronger antiviral defense. This makes normal cells more resistant to VSV infection.

However, tumor cells, which are often deficient in their interferon pathways (Stojdl, et al., Cancer Cell, 4:263-275 (2003); Wollmann, et al., J. Virol., 81:1479-1491 (2007)), largely remain susceptible to VSV oncolysis, even with M51 attenuation. VSV-M51 lacks some of VSV's inherent oncolytic potency in vivo, in part due to an effective activation of the systemic immune response to virally infected cells that can reduce the time interval during which VSV can act to infect tumors (Wu, et al., Hum. Gene Ther., 19:635-647 (2008)). In addition, whereas VSV, and probably first-position mutants, induce apoptosis through the caspase-independent mitochondrial pathway, VSV-M51 may induce apoptosis through a caspase-dependent pathway (Gaddy, et al., J. Virol., 79:4170-4179 (2005)), which may have consequences for antitumor targeting. Previous studies have shown that VSV M51 mutants are attenuated in normal cells but still infect many cancer cells. M51 mutants have been used to target brain cancer (Lun, et al, J. Natl. Cancer Inst., 98:1546-1557 (2006)).

4. Gene Switching and Rearrangement

Altering the order of genes can also be used to attenuate virus (Clarke, et al., J. Virol., 81:2056-2064, (2007), Cooper, et al., J. Virol., 82:207-219 (2008), Flanagan, et al., J. Virol., 75:6107-6114 (2001)). VSV is highly immunogenic, and a substantial B and T cell response from the adaptive immune system will ultimately limit VSV infection, which will halt runaway long-lasting viral infections. A virus that shows enhanced selectivity, and a faster rate of infection, will have a greater likelihood of eliminating cancer cells before the virus is eliminated by the immune system. However, the use of VSV against cancer cells does not have to be restricted to a single application. By molecular substitution of the G-protein for enhancing immune responses against foreign genes expressed by VSV, one could switch the original Indiana G protein of the virus with the G protein from VSV New Jersey or Chandipura, allowing a slightly different antigen presentation, and reducing the initial response of the adaptive immune system to second or third oncolytic inoculations with VSV.

It also may be desirable to rearrange the VSV genome. For example, shifting the L-gene to the sixth position, by rearrangement or insertion of an additional gene upstream, can result in attenuated L-protein synthesis and a slight reduction in replication (Dalton and Rose, Virology, 279(2):414-21 (2001)), an advantage when considering treatment of the brain.

5. Adaptive Passaging

Repeat passaging of virulent strains under evolutionary pressure can also be used to generate attenuated virus, increase specificity of the virus for a particular target cells, and/or increase the oncolytic potential of the virus. For example, VSV-rp30 (“30 times repeated passaging”) is a wild-type-based VSV with an enhanced oncolytic profile (Wollmann, et al., J. Virol. 79:6005-6022 (2005)). As described in WO10/080,909, VSV-rp30 has a preference for glioblastoma over control cells and an increased cytolytic activity on brain tumor cells.

C. Multiple Mechanisms of Attenuation in Combination

Attenuation of a virus can increase or decrease the oncolytic potential of a virus. As shown in the Examples, it has been discovered that the most promising oncolytic viruses have more than one attenuating characteristic. In the most preferred embodiments, the attenuated virus has at least two different molecular mechanisms of attenuation. In some embodiments, the virus has three or more attenuating characteristics.

Viruses generated from a DNA plasmid are substantively attenuated for virulence compared with wild-type VSV (Lawson, et al., Proc. Natl. Acad. Sci. USA, 92:4477-4481 (1995); Roberts, et al., J. Virol., 72:4704-4711 (1998)). Adding a transgene or reporter gene, such as sequences encoding the targeting or therapeutic proteins described below, to the viral genome also served to attenuate the resultant virus. As shown in the Examples below, this is particularly effective when the transgene is added at the first position, resulting in greater expression of the transgene than when it is placed in a secondary position, and also causing a reduction in the expression of all five of the viral structural genes (Clarke, et al., J. Virol., 81:2056-2064 (2007); Cooper, et al., J. Virol., 82:207-219 (2008); Ramsburg, et al., J. Virol., 79:15043-15-53 (2005); van den Pol, et al., J. Camp. Neurol., 516:456-481 (2009)).

First-position (p1) attenuated viruses are of particular interest for oncolysis. Insertion of a transgene, such as Green Fluorescent Protein (GFP), or Red Fluorescent Protein (RFP) reporter genes, results in virus that retains oncolytic capacity combined with reduced infection of normal cells (for example VSV-p1-GFP and VSV-p1-RFP). The nature of the transgene can also contribute to the attenuation of the virus. For example, as shown in the data presented below, the two fluorescent reporters are different in more than just color. The RFP (dsRed) combines to form a red tetramer, and this tetramer may have slightly greater toxicity than GFP (Long, et al., BMC Biotechnol., 5:20 (2005)). It is believed this reduces replication and budding of progeny VSV-p1-RFP and increases the toxicity of the virus when a cancer cell is infected. First position-VSV mutants are similarly attenuated, and show substantially reduced neurotoxicity after intranasal inoculation, but are still able to target glioblastoma in the brain after peripheral intravenous administration.

It may be desirable to switch or combine various substitutions, deletions, and insertions to further modify the phenotype of the virus. For example, an attenuated VSV can have both a truncation of the cytoplasmic tail of the G protein, and a deletion or mutation in the M protein. As described below, VSV-CT9-M51 is characterized by a truncation of the cytoplasmic tail of the G protein to 9 amino acids and a deletion of the fifty-first (51) amino acid of the M protein. VSV-CT9-M51 viruses may or may not, but preferably do contain a GFP reporter gene inserted between the G and L genes. The VSVCT9-M51 described in the examples below was constructed by Jack Rose's lab. It is derived from a recombinant version of the San Juan strain of Indiana serotype VSV, the genome of which consists of a single negative strand of RNA that encodes five genes, N, P, M, G and L. It has been discovered that viruses having both an M protein deletion, and trunctation of the cytoplasmic tail retain oncolytic activity, yet have reduced neurovirulence to normal cells.

It is believed that the molecular mechanism of attenuation is an important factor in the oncolytic potential of the virus. The Examples below are directed to the evaluation of ten specific attenuated oncolytic viruses. Of the 10 VSVs examined, four showed an optimal phenotype, including VSV-M51, VSV-CT9-M51, VSV-p1-GFP, and VSV-p1-RFP. The remaining VSVs tested either showed a limited ability to destroy tumor cells (VSV-dG-GFP, VSV-dG-RFP, and VSV-CT1) or did not show sufficiently attenuated virulence against normal cells (VSV-G/GFP, VSV-rp30, and VSV-CT9).

One important index of oncolytic potential is the ratio of viral replication in normal/control cells versus tumor or cancer cells. These ratios serve as an important index of the relative levels of viral replication in normal and tumor cells. A large ratio indicates greater replication in cancer cells than in control cells. In preferred embodiments, the ratio of replication of normal cells:target cells is greater than about 1:100, preferable greater than about 1:250, more preferable greater than about 1:500, most preferably great than about 1:1000. As shown in Example 1 below, the ratios for the ten viruses tested were: VSV-G/GFP, 1:100; VSV-rp30, 1:121; VSV-M51, 1:287; VSV-CT9-M51, 1:341; VSV-CT9, 1:237; VSV-CT1, 1:74; VSV-p1-GFP, 1:386; and VSV-p1-RFP, 1:602.

D. Viruses Engineered to Express Therapeutic or Targeting Proteins

Viruses may be modified to express one or more targeting or therapeutic proteins, separately or as a part of other expressed proteins. The viral genome of VSV has the capacity to accommodate additional genetic material. At least two additional transcription units, totaling 4.5 kb, can be added to the genome, and methods for doing so are known in the art. The added genes are stably maintained in the genome upon repeated passage (Schnell, et al., EMBO Journal, 17:1289-1296 (1998); Schnell, et al., PNAS, 93: 11359-11365 (1996); Schnell, et al., Journal of Virology, 70:2318-2323 (1996); Kahn, et al., Virology, 254, 81-91 (1999)).

Viruses can be engineered to include one or more additional genes that target the virus to cells of interest, see for example U.S. Pat. No. 7,429,481. In preferred embodiments, expression of the gene results in expression of a ligand on the surface of the virus containing one or more domains that bind to antigens, ligands or receptors that are specific to tumor cells, or are upregulated in tumor cells compared to normal tissue. Appropriate targeting ligands will depend on the cell or cancer of interest and will be known to those skilled in the art.

For example, virus can be engineered to bind to antigens or receptors that are specific to tumor cells or tumor-associated neovasculature, or are upregulated in tumor cells or tumor-associated neovasculature compared to normal tissue.

1. Therapeutic Proteins

Viruses can also be engineered to include one or more additional genes that encode a therapeutic protein. Suitable therapeutic proteins, such as cytokines or chemokines, are known in the art. Preferred cytokines include, but are not limited to, granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-10 (IL-10), interleukin-12 (IL-12), interleukin-15 (IL-15), interleukin-21 (IL-21), interferon alpha (IFNα), interferon beta (IFNβ), interferon gamma (IFNγ), and IGIF, and variants and fragments thereof. In the most preferred embodiment, the therapeutic protein is an interferon, such as interferon alpha.

Suitable chemokines include, but are not limited to, an alpha-chemokine or a beta-chemokine, including, but not limited to, a C5a, interleukin-8 (IL-8), monocyte chemotactic protein 1alpha (MIP1α), monocyte chemotactic protein 1 beta (MIPβ), monocyte chemoattractant protein 1 (MCP-1), monocyte chemoattractant protein 3 (MCP-3), platelet activating factor (PAFR), N-formyl-methionyl-leucyl-[³H]phenylalanine (FMLPR), leukotriene B₄, gastrin releasing peptide (GRP), RANTES, eotaxin, lymphotactin, IP10, I-309, ENA78, GCP-2, NAP-2 and MGSA/gro, and variants and fragments thereof.

2. Antigens, Ligands, and Receptors to Target

a. Tumor-Specific and Tumor-Associated Antigens

In one embodiment the viral surface contains a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are known.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, so these antigens are particularly preferred targets for oncotherapy and immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997)). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (FLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen mesothelin, defined by reactivity with monoclonal antibody K−1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K−1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession NO: U48722), HER2 (Yoshino, et al., J. Immunol., 152:2393 (1994); Disis, et al., Canc. Res., 54:16 (1994); GenBank Ace. Nos. X03363 and M17730), HER3 (GenBank Ace. Nos. U29339 and M34309), HER4 (Plowman, et al., Nature, 366:473 (1993); GenBank Ace. Nos. L07868 and T64105), epidermal growth factor receptor (EGFR) (GenBank Acc. Nos. U48722, and KO3193), vascular endothelial cell growth factor (GenBank NO: M32977), vascular endothelial cell growth factor receptor (GenBank Ace. Nos. AF022375, 1680143, U48801 and X62568), insulin-like growth factor-I (GenBank Ace. Nos. X00173, X56774, X56773, X06043, European Patent No. GB 2241703), insulin-like growth factor-II (GenBank Ace. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Ace, Nos. M38651, X03635, X99101, U47678 and M12674), progesterone receptor (GenBank Acc. Nos. X51730, X69068 and M15716), follicle stimulating hormone receptor (FSH-R) (GenBank Ace. Nos. Z34260 and M65085), retinoic acid receptor (GenBank Ace. Nos. L12060, M60909, X77664, X57280, X07282 and X06538), MUC-1 (Barnes, et al., Proc. Nat. Acad. Sci. USA, 86:7159 (1989); GenBank Ace. Nos. M65132 and M64928) NY-ESO-1 (GenBank Ace. Nos. AJ003149 and U87459), NA 17-A (PCT Publication NO: WO 96/40039), Melan-A/MART-1 (Kawakami, et al., Proc. Nat. Acad. Sci. USA, 91:3515 (1994); GenBank Ace. Nos. U06654 and U06452), tyrosinase (Topalian, et al., Proc. Nat. Acad. Sci. USA, 91:9461 (1994); GenBank Ace. NO: M26729; Weber, et al., J Clin. Invest, 102:1258 (1998)), Gp-100 (Kawakami, et al., Proc. Nat. Acad. Sci. USA, 91:3515 (1994); GenBank Ace. NO: S73003, Adema, et al., J. Biol. Chem., 269:20126 (1994)), MAGE (van den Bruggen, et al., Science, 254:1643 (1991)); GenBank Ace. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Ace. NO: U19180; U.S. Pat. Nos. 5,683,886 and 5,571,711), GAGE (GenBank Ace. Nos. AF055475, AF055474, AF055473, U19147, U19146, U19145, U19144, U19143 and U19142), any of the CIA class of receptors including in particular HOM-MEL-40 antigen encoded by the SSX2 gene (GenBank Ace. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Ace. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession NO: X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including, but not limited to, MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but are not limited to, alpha-actinin-4, Bcr-Abl fusion protein, Casp-8, beta-catenin, cdc27, cdk4, cdkn2a, coa-I, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/MeI-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791T 72, α-fetoprotein, 13HCG, BCA225, BTAA, CA 125, CA 15-3 (CA 27.29\CAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed viruses.

b. Antigens Associated with Tumor Neovasculature

Oncolytic viral therapeutics can be more effective in treating tumors by targeting to blood vessels of the tumor. Tumor-associated neovasculature provides a readily accessible route through which viral therapeutics can access the tumor. In one embodiment the viral proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed viruses.

c. Chemokines/Chemokine Receptors

In another embodiment, the virus is engineered to express a domain that specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups: CXC, CC, CX3C and C chemokines. The CXC chemokines can be further classified into ELR+ and ELR− chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

3. Molecular Classes of Targeting Domains

a. Ligands and Receptors

In one embodiment, tumor or tumor-associated neovasculature targeting domains are ligands that bind to cell surface antigens or receptors that are specifically expressed on tumor cells or tumor-associated neovasculature or are overexpressed on tumor cells or tumor-associated neovasculature as compared to normal tissue. Tumors also secrete a large number of ligands into the tumor microenvironment that affect tumor growth and development. Receptors that bind to ligands secreted by tumors, including, but not limited to, growth factors, cytokines and chemokines, including the chemokines discussed above, are suitable as targeting domains for the viruses disclosed herein. Ligands secreted by tumors can be targeted using soluble fragments of receptors that bind to the secreted ligands. Soluble receptor fragments are fragments of polypeptides that may be shed, secreted or otherwise extracted from the producing cells and include the entire extracellular domain, or fragments thereof.

b. Single Polypeptide Antibodies

In another embodiment, tumor or tumor-associated neovasculature targeting domains are single polypeptide antibodies that bind to cell surface antigens or receptors that are specifically expressed on tumor cells or tumor-associated neovasculature or are overexpressed on tumor cells or tumor-associated neovasculature as compared to normal tissue.

c. Fe Domains

In another embodiment, tumor or tumor-associated neovasculature targeting domains are Fc domains of immunoglobulin heavy chains that bind to Fc receptors expressed on tumor cells or on tumor-associated neovaseulature. As defined herein, the Fc region includes polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human or murine immunoglobulin. In a more preferred embodiment, the Fc domain is derived from human IgG1 or murine IgG2a including the C_{H}2 and C_{H}3 regions.

E. Pharmaceutical Carriers

Pharmaceutical compositions containing virus may be for systemic or local administration, such as intratumoral. Dosage forms for administration by parenteral (intramuscular (IM), intraperitoneal (IP), intravenous (IV) or subcutaneous injection (SC)), or transmucosal (nasal, vaginal, pulmonary, or rectal) routes of administration can be formulated.

In some in vivo approaches, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected. Therapeutically effective amounts of the viruses disclosed herein cause a reduction in tumor progression of reduction of tumor burden.

For the compositions disclosed herein and nucleic acids encoding the same, appropriate dosage levels for treatment of various conditions in various patients, can be determined by a person skilled in the art, considering the therapeutic context, age, and general health of the recipient. The selected dosage depends upon the desired therapeutic effect, on the route of administration, and on the duration of the treatment desired. Active virus can also be measured in terms of plaque-forming units (PFU). A plaque-forming unit can be defined as areas of cell lysis (CPE) in monolayer cell culture, under overlay conditions, initiated by infection with a single virus particle. Generally dosage levels of virus between 10² and 10¹² PFU are administered to humans. Virus is typically administered in a liquid suspension, in a volume ranging between 10 μl and 100 ml depending on the route of administration. The dose may be administered once or multiple times. Virus delivered locally, such as by intratumoral injection, is typically administered in lower doses than virus administered systemically. When administered locally, therapeutic virus is administered to humans at dosage levels between 10² and 10⁶ PFU. Pharmaceutical dosage units of virus are typically administered as a liquid suspension, in a low volume. The volume for local administration can range from about 20n1 to about 2000. Typically the dose for local administration will be about 100 μl delivered intratumorly in multiple doses. For systemic or regional administration via subcutaneous, intramuscular, intra-organ, or intravenous administration the dosage will typically be from about 0.5 ml to 100 ml.

Actual dosage, or viral titer will depend on the oncolytic activity of the virus. For attenuated viruses with increased oncolytic activity, for example, viruses exhibiting lower cytotoxicity for normal cells, a patient may be able to tolerate a high viral titer for example between about 10⁷ and 10¹², or more for systemic administration, or between about 10⁴ and 10⁶ or more for local administration. For attenuated viruses exhibiting increased cytotoxicity for target cells, such as tumor or cancer cells, it may be desirable to administer a low viral titer for example between about 10² and 10⁶, or less for systemic administration, or between about 10² and 10⁴, or less for local administration. The most desirable virus will have high specific activity (i.e. infectivity) for tumor cells, and low cytotoxicity toward normal cells. Therefore, when possible, lower effective dosages are preferred to reduce toxicity to normal cells.

The term “pharmaceutically acceptable” means a non-toxic material that does not interfere with the effectiveness of the biological activity of the active ingredients. The term “pharmaceutically-acceptable carrier” means one or more compatible solid or liquid fillers, diluents or encapsulating substances which are suitable for administration to a human or other vertebrate animal. The term “carrier” refers to an organic or inorganic ingredient, natural or synthetic, with which the active ingredient is combined to facilitate the application.

Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, thickening agents, co-solvents, adhesives, antioxidants, buffers, viscosity and absorption enhancing agents and agents capable of adjusting osmolarity of the formulation. Proper formulation is dependent upon the route of administration chosen. If desired, the compositions may also contain minor amount of nontoxic auxiliary substances such as wetting or emulsifying agents, dyes, pH buffering agents, or preservatives. The formulations should not include membrane disrupting agents which could kill or inactivate the virus.

1. Formulations for Local or Parenteral Administration

In a preferred embodiment, compositions including oncolytic virus disclosed herein, are administered in an aqueous solution, by parenteral injection. Injection includes, but it not limited to, local, intratumoral, intravenous, intraperitoneal, intramuscular, or subcutaneous. The formulation may also be in the form of a suspension or emulsion. In general, pharmaceutical compositions are provided including effective amounts of virus, and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as anti-oxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. A preferred solution is phosphate buffered saline or sterile saline.

2. Formulations for Mucosal Administration

In some embodiments, the compositions are formulated for mucosal administration, such as through nasal, pulmonary, or buccal delivery.

Mucosal formulations may include one or more agents for enhancing delivery through the nasal mucosa. Agents for enhancing mucosal delivery are known in the art, see for example U.S. Patent Application No. 20090252672 to Eddington, and U.S. Patent Application No. 20090047234 to Touitou. Acceptable agents include, but are not limited to, chelators of calcium (EDTA), inhibitors of nasal enzymes (boro-leucin, aprotinin), inhibitors of muco-ciliar clearance (preservatives), solubilizers of nasal membrane (cyclodextrin, fatty acids, surfactants) and formation of micelles (surfactants such as bile acids, Laureth 9 and taurodehydrofusidate (STDHF)). Compositions may include one or more absorption enhancers, including surfactants, fatty acids, and chitosan derivatives, which can enhance delivery by modulation of the tight junctions (TJ) (13. J. Aungst, et al., J. Pharm. Sci. 89(4):429-442 (2000)). In general, the optimal absorption enhancer should possess the following qualities: its effect should be reversible, it should provide a rapid permeation enhancing effect on the cellular membrane of the mucosa, and it should be non-cytotoxic at the effective concentration level and without deleterious and/or irreversible effects on the cellular membrane, virus membrane, or cytoskeleton of the TJ.

F. Kits

Dosage units include virus in a pharmaceutically acceptable carrier for shipping and storage and/or administration. Active virus should be shipped and stored using a method consistent with viability such as in cooler containing dry ice so that cells are maintained below 4° C., and preferably below −20° C. VSV virus should not be lyophilized. Components of the kit may be packaged individually and can be sterile. In one embodiment, a pharmaceutically acceptable carrier containing an effective amount of virus is shipped and stored in a sterile vial. The sterile vial may contain enough virus for one or more doses. Virus may be shipped and stored in a volume suitable for administration, or may be provided in a concentrated titer that is diluted prior to administration. In another embodiment, a pharmaceutically acceptable carrier containing an effective amount of virus can be shipped and stored in a syringe.

Typical concentrations of viral particles in the sterile saline, phosphate buffered saline or other suitable media for the virus is in the range of 10⁸ to 10⁹ with a maximum of 10¹². Dosage units should not contain membrane disruptive agents nor should the viral solution be frozen and dried (i.e., lyophilized), which could kill the virus.

Kits containing syringes of various capacities or vessels with deformable sides (e.g., plastic vessels or plastic-sided vessels) that can be squeezed to force a liquid composition out of an orifice are provided. The size and design of the syringe will depend on the route of administration. For example, in one embodiment, a syringe for administering virus intratumorally, is capable of accurately delivering a smaller volume (such as 1 to 100 μl). Typically, a larger syringe, pump or catheter will be used to administer virus systemically.

The kits optionally include one or more of the following: bioactive agents, media, excipients and one or more of: a syringe, a bandage, a disinfectant, a local anesthetic, an analgesic agent, surgical thread, scissors, a sterile fluid, and a sterile vessel. Kits for intranasal administration may optionally contain a delivery device for facilitating intranasal delivery, such as a nasal sprayer. The kits are generally provided in a container, e.g., a plastic, cardboard, or metal container suitable for commercial sale. Any of the kits can include instructions for use.

### III. Methods of Determining Enhanced Oncogenic Potential

An important consideration in the design of an oncolytic viral therapy, is toxicity to normal cells. It is highly desirable to identify oncolytic viruses with high specificity, infectivity, and cytotoxicity toward tumor cells, and low or no specificity, infectivity, or cytotoxicity toward normal cells. It has been discovered that attenuation of viruses can result in improved specificity of oncolyic viruses for tumor cells, particular brain tumor cells, when compared to normal, non-tumor cells. As illustrated in the Examples below, in vitro and in vivo tests can be used to identify viruses with improved oncolytic potential and safety profile compared to wildtype or other attenuated, or recombinant viruses.

A. Viral infection and cytopathic effects

Viral infection and the cytopathic effects of attenuated viruses can be determined in vitro using cultured tumor cells, such as gliablastoma cells, and non-tumor control cells, such as normal glia cells. Normal and tumor cells are cultured in parallel according cell specific conditions that are known in the art. After the cultures are established, fresh medium containing virus is added. Typically, viral infection assays will include a viral titer characterized by a low multiplicity of infection [MOI], however the MOI can be varied. Multiplicity of infection refers to the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a tissue culture well divided by the number of target cells present in that well. A low MOI helps in assessing infectivity at a low dose, because viral replication is required to have an effect on a great number of tumor cells. The MOI is preferably <10, more preferably <1, more preferably about 0.5, and most preferably about 0.1 when assessing the infectivity and cytopathic effects of viruses.

Cultures can be observed for period of time post infection, for example 3 days dpi (days post infection). Infectivity can be monitored by any suitable method known in the art, for example, by monitoring the morphology of cells for cytopathic effects by light microscopy and, or identification of infectious virus by electron microscopy. If the subject virus is a virus engineered to express a reporter construction, such as GFP, expression of the construct can be monitored by a means of detecting expression of the reporter construct, for example by fluorescent microscopy. Cells can also be fixed and stained using immunohistochemical techniques.

B. Cell Growth and Viability

Cell viability can also be monitored by methods known in the art. For example, terminal deoxynucleotidyl transferase dUTP nick end labeling (TUNEL) or detection of caspases by immunohistochemistry can be used to assess apoptosis. However, it is believed that some virus-induced cell death is caspase-dependent, while other virus-induced cell death is caspase-independent, therefore the mechanism of cell death should be consider in selecting a cell viability assay. As described in the Examples below, the MTT assay and the MTS assay are colorimetric assays for measuring the activity of enzymes that reduce MTT (3-(4,5-Dimethylthiazol-2-yl)-2,5-diphenyltetrazolium bromide, a yellow tetrazole) or close dyes (XTT, MTS, WSTs) to formazan dyes, giving a purple color. These assays can be used to assess viability (cell counting) and the proliferation of cells (cell culture assays). They can also be used to determine cytotoxicity of potential medicinal agents and toxic materials, for example oncolytic viruses, since those agents would stimulate or inhibit cell viability and growth.

C. Viral Replication

Local self-amplification is one of the mainstays of replication competent oncolytic viruses. Preferably, oncolytic viruses selectively replicate faster or more efficiently in tumor cells than normal cells. Viral replication can be determined using standard plaque assay techniques that are known in the art. For example, as described in the Example 2 below, a monolayer of cells can be infected with virus, and supernatant collected and analyzed for viral titer at various time points post-infection.

These assays can be used to establish a semiquantitative measure of relative viral replication in control versus tumor cells, i.e., the ratio of replication. Larger ratios are indicative of preferred viral candidates, namely, viruses that replicated more efficiently in cancer cells than in noncancer cells. As described in the examples below, for the specific viruses disclosed herein, the largest ratios were 1:386 and 1:602, for VSV-p1-GFP and VSV-p1-RFP, respectively. These contrasted with relatively less effective oncolytic performers, such as VSV-CT1, which had a ratio of 1:74 and was relatively ineffective at killing glioblastoma cells.

Infectivity, cytotoxicity, cell growth and viability, and virus replication can also be tested in the presence or absence of prophylactic or therapeutic agents. For example, as described in the Examples below, INF-α has a protective effect on normal cells, without protecting tumor cells against oncolytic infection by some VSV viruses. Therefore, it may be beneficial to test these parameters of oncolytic performance in the presence of INF-α.

### IV. Methods of Use

A. Subjects to be Treated

In general, the compositions are useful for targeting and destroying a cell or cells of interest. In a preferred embodiment, the cells of interest are cancer cells. For example, compositions are useful as therapeutic compositions, which can be used to treat benign or malignant tumors.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer typically refers to a malignant tumor. In addition to uncontrolled growth, malignant tumors can exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The examples below demonstrate that the VSV virus disclosed herein are oncolytic to tumors in vitro or in vivo.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. The disclosed compositions are particularly effective in treating carcinomas. Sarcomas, which arise less frequently, are derived from mesodermal connective tissues such as bone, fat, and cartilage. VSV is selective for sarcomas compared to normal mesoderm. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, tumors arising from cancers such as vascular cancer such as multiple myeloma, adenocarcinomas and sarcomas, of bone, bladder, brain, breast, cervical, colo-rectal, esophageal, kidney, liver, lung, nasopharangeal, pancreatic, prostate, skin, stomach, and uterine. In some embodiments, the disclosed compositions are used to treat multiple tumors or cancer types concurrently. The compositions can also be used to treat metastases or tumors at multiple locations. As shown in the examples below, the disclosed, attenuated oncolytic viruses are particularly effective in treating gliomas (including astrocytomas) in the brain. In some embodiments, the composition is used to treat lung or breast cancer carcinomas, which are the source of many brain cancers. In some embodiments, the disclosed compositions are used to treat multiple cancer types concurrently. The compositions can also be used to treat metastases or tumors at multiple locations.

The disclosed compositions and methods are particularly useful in treating brain tumors. Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells), lymphatic tissue, blood vessels), in the cranial nerves, in the brain envelopes (meninges), skull, pituitary and pineal gland, or spread from cancers primarily located in other organs (metastatic tumors). “Primary” brain tumors originate in the brain and “secondary” (metastatic) brain tumors originate from cancer cells that have migrated from other parts of the body. Primary brain cancer rarely spreads beyond the central nervous system, and death results from uncontrolled tumor growth within the limited space of the skull. Metastatic brain cancer indicates advanced disease and has a poor prognosis. Primary brain tumors can be cancerous or noncancerous. Both types take up space in the brain and may cause serious symptoms (e.g., vision or hearing loss) and complications (e.g., stroke). All cancerous brain tumors are life threatening (malignant) because they have an aggressive and invasive nature. A noncancerous primary brain tumor is life threatening when it compromises vital structures (e.g., an artery).

Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells), lymphatic tissue, blood vessels), in the cranial nerves, in the brain envelopes (meninges), skull, pituitary and pineal gland, or spread from cancers primarily located in other organs (metastatic tumors). Examples of brain tumors include, but are not limited to oligodendroglioma, meningioma, supratentorial ependymona, pineal region tumors, medulloblastoma, cerebellar astrocytoma, infratentorial ependymona, brainstem glioma, schwannomas, pituitary tumors, craniopharyngioma, optic glioma, and astrocytoma. In the most preferred embodiment, a composition containing an attenuated oncolytic VSV is used for treating glioblastoma.

VSV has a good oncolytic profile, in-part, by taking advantage of defects in the innate cellular anti-viral defense system, which is a common feature in malignancies, including colon, breast, prostate, liver, and leukemia. Reduction in interferon-related antiviral defenses enhance infection of cancer cells by attenuated VSV viruses. Activation of the interferon pathway protects normal human brain cells from VSV infection while maintaining the vulnerability of human glioblastoma cells to viral destruction (Wollmann, et al. J. Virol., 81(3):1479-1491 (2007)). In some embodiments, the disclosed compositions and methods are used to treat a population of cells with defects in the interferon system. In preferred embodiments, the cells with a defective interferon system or defective antiviral defense system are tumor cells that are susceptible to VSV infection and destruction in the presence of exogenous interferons such as IFN-α, or IFN-α/β pathway inducer polyriboinosinic polyribocytidylic acid [poly(I:C)].

B. Methods of Administration

Any acceptable method known to one of ordinary skill in the art may be used to administer a formulation to the subject. Preferably, administration of the formulations may be accomplished by any acceptable method which allows an effective amount of the oncolytic virus to reach their target. As generally used herein, an “effective amount” is that amount which is able to induce a desired result in a treated subject. The desired results will depend on the disease or condition to be treated. For example, in treating a subject with a tumor, in one embodiment, an effective amount of the composition reduces or stops tumor progression or at least reduces one or more symptoms of the tumor. Symptoms of cancer may be physical, such as tumor burden, or biological such as proliferation of cancer cells. The actual effective amounts of virus can vary according to factors including the specific virus administered, the particular composition formulated, the mode of administration, and the age, weight, condition of the subject being treated, as well as the route of administration and the disease or disorder.

The particular mode of administration selected will depend upon factors such as the particular formulation, the severity of the state of the subject being treated, and the dosage required to induce an effective response. The administration may be localized (i.e., to a particular region, physiological system, tissue, organ, or cell type) or systemic. The compositions can be administered by a number of routes including, but not limited to, injection: intravenous, intraarterial, intraperitoneal, intramuscular, or subcutaneous, or to a mucosal surface (oral, sublingual or buccal, nasal, rectal, vaginal, pulmonary) and special means such as convection enhanced delivery. In a preferred embodiment, the oncolytic virus is administered in an aqueous solution, by parenteral injection. In one embodiment, the composition is injected locally at the site of treatment, such as a tumor. For example, treatment of brain tumors may include intercanial injection of a composition containing oncolytic virus directly into the tumor. In some embodiments, the composition is delivered systemically, by injection into the circulatory system (i.e. intravenous) or an appropriate lymphoid tissue, such as the spleen, lymph nodes or mucosal-associated lymphoid tissue. The injections can be given at one, or multiple locations. In a preferred embodiment, one treatment is sufficient. In some embodiments, multiple treatments are required.

The composition can also be administered mucosally. One example of mucosal administration is intranasal delivery. Intranasal administration can result in systemic or local delivery of oncolytic virus. For example, following intranasal delivery, virus gain access to the CNS through the olfactory nerve, which projects to the glomeruli in the olfactory bulb of the brain (van den Poi et al., J. Virol, 76:1309-27 (2002)).

C. Combination Therapies

Administration of the disclosed compositions containing oncolytic viruses may be coupled with surgical, radiologic, other therapeutic approaches to treatment of cancer.

1. Surgery

The disclosed compositions and methods can be used as an adjunct to surgery. Surgery is a common treatment for many types of benign and malignant tumors. As it is often not possible to remove all the tumor cells during surgery, the disclosed compositions containing oncolytic virus are particularly useful subsequent to resection of the primary tumor mass, and would be able to infect and destroy even dispersed tumor cells.

An additional situation where an oncolytic virus may be helpful is in regions where the tumor is either wrapped around critical vasculature, or in an area that is difficult to treat surgically. Widely disseminated metastatic carcinomas are also a potential target given the high efficiency of VSV against many systemic malignancies such as breast, prostate, liver or colon carcinomas or lymphomas (Stojdl, et al., Cancer Cell, 4:263-275 (2003); Ahmed, Virology, 330:34-49 (2004); Ebert, et al., Cancer Gene Ther., 12:350-358 (2005); Shinozaki, et al., Hepatology, 41:196-203 (2005); Lichty, et al., Hum. Gene Ther., 15:821-831 (2004)).

In a preferred embodiment, the disclosed compositions and methods are used as an adjunct or alternative to neurosurgery. The compositions are particularly well suited to treat areas of the brain that is difficult to treat surgically, for instance high grade tumors of the brain stem, motor cortex, basal ganglia, or internal capsule. High grade gliomas in these locations are generally considered inoperable.

2. Therapeutic Agents

The viral compositions can be administered to a subject in need thereof alone or in combination with one or more additional therapeutic agents selected based on the condition, disorder or disease to be treated. A description of the various classes of suitable pharmacological agents and drugs may be found in Goodman and Gilman, The Pharmacological Basis of Therapeutics, (11th Ed., McGraw-Hill Publishing Co.) (2005).

Additional therapeutic agents include conventional cancer therapeutics such as chemotherapeutic agents, cytokines, chemokines, and radiation therapy. The majority of chemotherapeutic drugs can be divided into: alkylating agents, antimetabolites, anthracyclines, plant alkaloids, topoisomerase inhibitors, and other antitumour agents. All of these drugs affect cell division or DNA synthesis and function in some way. Additional therapeutics include monoclonal antibodies and the tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

Preferred chemotherapeutics will affect tumors or cancer cells, without diminishing the activity of the virus. For example, in a preferred embodiment, the additional therapeutic agent inhibits proliferation of cancer cells without affecting targeting, infectivity, or replication of the virus.

a. Immunosuppressants

It may be desirable to administer viral compositions in combination with an immunosuppressant. Oncolytic viruses such as VSV are highly immunogenic, and a substantial B and T cell response from the adaptive immune system would ultimately limit viral infection. An immunosuppressant attenuates the host immune response and prolongs viral infection. Immunosuppressants are known in the art and include glucocorticoids, cytostatics (such as alkylating agents, antimetabolites, and cytotoxic antibodies), antibodies (such as those directed against T-cell recepotors or Il-2 receptors), drugs acting on immunophilins (such as cyclosporine, tacrolimus, and sirolimus) and other drugs (such as interferons, opioids, TNF binding proteins, mycophenolate, and other small molecules such as fingolimod). The dosage ranges for immunosuppressant agents are known in the art. The specific dosage will depend upon the desired therapeutic effect, the route of administration, and on the duration of the treatment desired. For example, when used as an immunosuppressant, a cytostatic maybe administered at a lower dosage than when used in chemotherapy. Suitable immunosuppressants include, but are not limited to, FK506, prednisone, methylprednisolone, cyclophosphamide, thalidomide, azathioprine, and daclizumab, physalin B, physalin F, physalin G, seco-steroids purified from Physalis angulata L., 15-deoxyspergualin, MMF, rapamycin and its derivatives, CCI-779, FR 900520, FR 900523, NK86-1086, depsidomycin, kanglemycin-C, spergualin, prodigiosin25-c, cammunomicin, demethomycin, tetranactin, tranilast, stevastelins, myriocin, gliotoxin, FR 651814, SDZ214-104, bredinin, WS9482, mycophenolic acid, mimoribine, misoprostol, OKT3, anti-IL-2 receptor antibodies, azasporine, leflunomide, mizoribine, azaspirane, paclitaxel, altretamine, busulfan, chlorambucil, ifosfamide, mechlorethamine, melphalan, thiotepa, cladribine, fluorouracil, floxuridine, gemcitabine, thioguanine, pentostatin, methotrexate, 6-mercaptopurine, cytarabine, carmustine, lomustine, streptozotocin, carboplatin, cisplatin, oxaliplatin, iproplatin, tetraplatin, lobaplatin, JM216, JM335, fludarabine, aminoglutethimide, flutamide, goserelin, leuprolide, megestrol acetate, cyproterone acetate, tamoxifen, anastrozole, bicalutamide, dexamethasone, diethylstilbestrol, bleomycin, dactinomycin, daunorubicin, doxirubicin, idarubicin, mitoxantrone, losoxantrone, mitomycin-c, plicamycin, paclitaxel, docetaxel, topotecan, irinotecan, 9-amino camptothecan, 9-nitro camptothecan, GS-211, etoposide, teniposide, vinblastine, vincristine, vinorelbine, procarbazine, asparaginase, pegaspargase, octreotide, estramustine, and hydroxyurea, and combinations thereof. Preferred immunosuppressants will preferentially reduce or inhibit the subject's immune response, without reducing or inhibiting the activity of the virus. For example, in a preferred embodiment, the additional therapeutic agent inhibits activation and/or proliferation of the tumor cells without affecting targeting, infectivity, or replication of the virus.

b. Anticancer Agents

The compositions can be administered with an antibody or antigen binding fragment thereof specific for growth factor receptors or tumor specific antigens. Representative growth factors receptors include, but are not limited to, epidermal growth factor receptor (EGFR; HER1); c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factorAreceptor (CSF-1R) (c-Fms); steel receptor (c-Kit); Flk2/Flt3; fibroblast growth factor receptor 1 (Flg/Cek1); fibroblast growth factor receptor 2 (Bek/Cek3/K-Sam); Fibroblast growth factor receptor 3; Fibroblast growth factor receptor 4; nerve growth factor receptor (NGFR) (TrkA); BDNF receptor (TrkB); NT-3-receptor (TrkC); vascular endothelial growth factor receptor 1 (Flt1); vascular endothelial growth factor receptor 2/Flk1/KDR; hepatocyte growth factor receptor (HGF-R/Met); Eph; Eck; Eek; Cek4/Mek4/HEK; CekS; Elk/Cek6; Cek7; Sek/Cek8; Cek9; Cek10; HEK11; 9 Ror1; Ror2; Ret; Axl; RYK; DDR; and Tie.

In some cases, cancer cells are resistant to infection by viruses such as VSV. For example, synovial sarcoma is shown to be highly resistant to infection by VSV, human cytomegalovirus, and Sindbis virus due to heightened basal expression of interferon-stimulated genes. For these viruses, an anticancer agent that attenuates interferon, such as valporoate, Jak1 inhibitor, or vaccinia virus B18R protein, may be used to enhance susceptibility of the cancer to treatment with attenuated, oncolytic VSV.

c. Therapeutic Proteins

It may be desirable to administer the disclosed compositions in combination with therapeutic proteins. VSV is an effective oncolytic virus, in-part, by taking advantage of defects in the interferon system. Administration of therapeutic proteins such as IFN-α, or IFN-α/β pathway inducer polyriboinosinic polyribocytidylic acid [poly(I:C)] are effective in protecting normal cells from the oncolytic activity, while leaving the tumor cells susceptible to infection and death (Wollmann, et al. J. Virol., 81(3): 1479-1491 (2007), Wollmann, et al., J. Virol, (2009)). Therefore, in some embodiments, the disclosed compositions are administered in combination with a therapeutic protein to reduce infectivity and death of normal cells. Suitable therapeutic proteins are described above.

d. Peripheral Immunization

It may be desirable to administer the disclosed compositions after peripheral immunization with the virus. Evidence shows that peripheral activation of the systemic immune system can protect the brain from VSV damage (Ozduman, et al., J. Virol., 83(22):11540-11549 (2009), and PCT/US2010/048472). Immunization is carried out first, preferably by intranasal or intramuscular delivery, or combination thereof. Immunization is followed by administration of oncolytic VSV virus for example by systemic or local administration.

### V. Methods of Manufacture

A. Engineering Recombinant VSV Viruses

The VSV genome is a single negative-sense, non-segmented stand of RNA that contains five genes (N, L, P, M, and G) and has a total size of 11.161 kb. Methods of engineering recombinant viruses by reconstituting VSV from DNA encoding a positive-sense stand of RNA are known in the art (Lawson, et al., PNAS, 92:4477-4481 (1995), Dalton and Rose, Virology, 279:414-421 (2001)). For example, recombinant DNA can be transcribed by T7 RNA polymerase to generate a full-length positive-strand RNA complimentary to the viral genome. Expression of this RNA in cells also expressing the VSV nucleocapsid protein and the two VSV polymerase subunits results in production of VSV virus (Lawson, et al., PNAS, 92:4477-4481 (1995)). In this way, VSV viruses can be engineered to create attenuated viruses, express variant proteins, additional proteins, foreign antigens, targeting proteins, or therapeutic proteins using known cloning methods.

B. Creating Mutant VSV Virus

RNA viruses are prone to spontaneous genetic variation. The mutation rate of VSV is about 10⁻⁴ per nucleotide replicated, which is approximately one nucleotide change per genome (Drake, et al., Proc. Natl. Acad. Sci. USA, 96:13910-13913). Therefore, mutant VSV viruses exhibiting desired properties can be developed by applying selective pressure. Methods for adaption of VSV viruses through repeated passaging is described in the art. See, for example, Wollmann, et al., J. Virol., 79(10): 6005-6022 (2005). Selective pressure can be applied by repeated passaging and enhanced selection to create mutant virus with desirable traits such as increased infectivity and oncolytic potential for a cell type of interest. The cell type of interest could be general, such as cancer cells, or specific such as glioblastoma cells. Mutant virus can also be selected based on reduced toxicity to normal cells. Methods of enhanced selection include, but are not limited to, short time for viral attachment to cells, collection of early viral progeny, and preabsorption of viral particles with high affinity of undesirable cells (such as normal cells). Mutations can be identified by sequencing the viral genome, for instance as described in Example 4 below.

DNA encoding the VSV genome can also be used as a substrate for random or site directed mutagenesis to develop VSV mutant viruses. Mutagenesis can be accomplished by a variety of standard, mutagenic procedures. Changes in single genes may be the consequence of point mutations that involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of nucleic acid replication or the movement of transposable genetic elements (transposons) within the genome. They also are induced following exposure to chemical or physical mutagens. Such mutation-inducing agents include ionizing radiations, ultraviolet light and a diverse array of chemicals such as alkylating agents and polycyclic aromatic hydrocarbons all of which are capable of interacting either directly or indirectly (generally following some metabolic biotransformations) with nucleic acids. The nucleic acid lesions induced by such environmental agents may lead to modifications of base sequence when the affected DNA is replicated or repaired and thus to a mutation, Mutation also can be site-directed through the use of particular targeting methods. Various types of mutagenesis such as random mutagenesis, e.g., insertional mutagenesis, chemical mutagenesis, radiation mutagenesis, in vitro scanning mutagenesis, random mutagenesis by fragmentation and reassembly, and site specific mutagenesis, e.g., directed evolution, are described in U.S. Patent Application No. 2007/0026012.

Mutant viruses can be prepared by site specific mutagenesis of nucleotides in the DNA encoding the protein, thereby producing DNA encoding the mutant. Techniques for making substitution mutations at predetermined sites in DNA having a known sequence are well known, for example M13 primer mutagenesis and PCR mutagenesis. Amino acid substitutions are typically of single residues, but can occur at a number of different locations at once. Insertions usually will be on the order of about from 1 to about 10 amino acid residues; and deletions will range about from 1 to about 30 residues, however insertions and deletions of a greater number of amino acids area also contemplated. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place.

### EXAMPLES

### Example 1

### VSV Variants Infect and Kill Brain Tumor Cells

Materials and Methods

Viruses

Ten (10) recombinant VSV variants were compared for both oncolytic capabilities and normal brain cell attenuation. The VSV variants used represent a systematic comparison of attenuated rVSVs. VSV-M51 is characterized by a codon deletion of methionine at the fifty-first position of the M protein, which reduces the viral suppression of cellular immunity against VSV (Ahmed, et al., Virology, 237:378-388 (1997); Coulon, et al., J. Gen. Virol., 71:991-996 (1990); Stojdl, et al., Cancer Cell, 4:263-275 (2003)).

VSV-CT1 and VSV-CT9 are characterized by mutations shortening the 27-amino-acid chain of the cytoplasmic G protein tail down to 1 and 9 amino respectively (Publicover, et al., J. Virol., 78:9317-9324 (2004)). Reducing the length of the cytoplasmic G protein reduces virulence (Schnell, et al., EMBO J., 17:1289-1296 (1998)). Shifting the order of the genes downward has also been reported to reduce virulence (Clarke, et al., J. Virol., 81:2056-2064 (2007); Flanagan, et al., J. Virol., 75:6107-6114 (2001)).

VSV-p1-GFP and VSV-p1-RFP, gene order shifting variants, are characterized by a wild-type-related genome that is shifted by the insertion of the GFP (or RFP) reporter gene at position 1 of the gene order. As a result, all other virus genes are moved downward, to positions 2 to 6.

VSV-dG-GFP and VSV-dG-RFP are gene deletion variants characterized insertion of the GFP (or RFP) reporter gene at position 1 of the gene order and by deletion of the entire G protein encoding sequence. Eliminating the G gene blocks the ability of the virus to infect cells; however, by adding the G protein in trans, as was done in the Examples described here, by generating the virus in cells that express the VSV-G protein (Publicover, et al., J. Virol., 79:13231-13238 (2005)), the replication-restricted viruses VSV-dG-GFP and VSV-dG-RFP will at least infect a single round of cells.

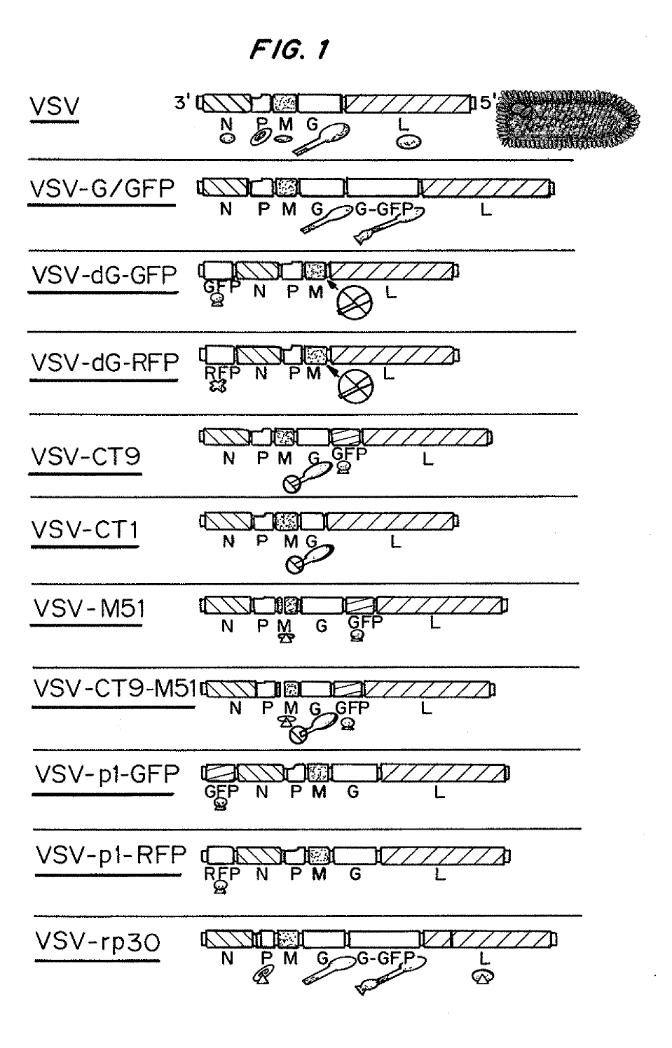

VSV-CT9-M51 is characterized by multiple attenuating mutations, including the M51 amino acid deletion and G protein CT-9 truncation described above (Publicover, et al., J. Virol., 80:7028-7036 (2006); van den Pol, et al., J. Comp. Neurol., 516:456-481 (2009)). VSV-rp30 is a glioma-passage-adapted VSV variant characterized by two amino acid changes, a S126L substitution in the VSV P protein and a D223Y substitution in the L protein. VSV-rp30 was generated from VSV-G/GFP through repeated passage and adaptation to glioblastoma cells, as previously reported (Wollmann, et al., J. Virol. 79:6005-6022 (2005)). The sequences of VSV-G/GFP and VSV-rp30 are disclosed in WO 2010/080909. The VSV-rp30 phenotype displayed enhanced infectivity and oncolytic activity. The reference virus for this comparative study was VSV-G/GFP, a recombinant VSV that was generated from cDNA, using sequence fragments from wild-type VSV Indiana strains (Dalton, et al., Virology, 279:414-421 (2001); Roberts, et al., J. Virol., 72:4704-4711 (1998); van den Pol., et al., J. Virol., 76:1309-1327 (2002)). VSV-G/GFP is characterized by an extra copy of the G protein fused to a GFP reporter gene downstream of the original G gene (Lawson, et at., Proc. Natl. Acad. Sci. USA, 92:4477-4481 (1995)). Though closely related to wild-type VSV, VSV-G/GFP has reduced virulence (Rose, et al., Cell, 106:359-549 (2001)). A schematic overview of the different VSV types, with their respective variations from the wild type, is displayed in FIG. 1.

Titers for all VSV variants were determined through plaque assays on BHK cells prior to experiments.

### Human Cells

The human glioblastoma cell line U87MG was obtained from ATCC (Manassas, Va.). These cells were stably transfected with the gene coding for monomeric dsRed, allowing easy detection of red human glioblastomas transplanted into mouse brains (see below) (Oezduman, et al., J. Neurosci., 28:1882-1893 (2008)). The U-118, U-373, and A-172 cell lines were kindly provided by R. Matthews (Syracuse, N.Y.). Normal human glia cells were established from tissue derived from surgery specimens from patients undergoing epilepsy surgery. Glia cell cultures were isolated through explant cultures and tested for immunoreactivity to glial fibrillary acidic protein (GFAP). Human cell preparation and use were approved by the Yale University Human Investigation Committee. All cells were kept in a humidified atmosphere containing 5% CO₂ at 37° C. U87 cells were fed with minimal essential medium (MEM) supplemented with 10% fetal bovine serum, 1% sodium pyruvate, and 1% nonessential amino acids. Normal human glia cells were propagated with MEM supplemented with 10% fetal bovine serum.

Viral Infection and Cytopathic Effects

For assessing infection and the appearance of cytopathic effects, cells were seeded in 12-well dishes at a density of 100,000 cells per dish in triplicate for each condition. After 12 h, fresh medium was added to each dish, containing 10⁴ PFU (multiplicity of infection [MOI]=0.1) of any of the 10 VSV variants. Cultures were observed for 3 days postinfection (dpi). GFP was monitored with an Olympus IX 71 fluorescence microscope, using a 485-nm excitation filter. Photomicrographs were taken with a Spot RT digital camera (Diagnostic Instruments, Sterling Heights, Mich.) interfaced with an Apple Macintosh computer. Contrast and color of the photomicrographs were adjusted with Adobe Photoshop.

Cell Growth and Viability

U87 and human glia control cells were plated in 96-well dishes at a density of 10,000 per well, using colorless MEM without phenol red. After 12 h, the medium was replaced with either fresh medium or medium containing 100 IU alpha interferon (IFN-α; Sigma-Aldrich) for 6 h of preincubation before the addition of 5,000 PFU of the indicated VSV variants. Viability was assessed using an MTT (Molecular Probes) assay according to the manufacturer's instructions. Optical density was read at 570 nm, using a Dynatech MR500 enzyme-linked immunosorbent assay (ELISA) plate reader (Dynatech Lab Inc, Alexandria, Va.), and corrected with background control subtraction. Each condition was tested in triplicate.

### Results

Since the VSV variants used display features of attenuation, the extent to which this attenuation might impair the oncolytic strength was examined. Fluorescence microscopy to detect expression of the GFP reporter gene in infected cells, phase-contrast microscopy to assess the presence of cytopathic effects, and MTT assay for quantification of cell viability and oncolytic capacity. Previous studies have shown a defective interferon response in cancer cells to be a main factor in selective VSV oncolysis. On the other hand, IFN provides protection against VSV to normal cells (Perry, et al., Cell Res., 5:407-422 (2005)). The effect of IFN of the infectivity of attenuated VSV was tested, and described below.

In the first set of experiments, U87 human glioblastoma cells were infected at an MOI of <0.1, and signs of infection were observed over the course of 2 days. One of the main advantages of replication-competent oncolytic viruses over replication-deficient vectors is the local self-amplification of the therapeutic effect, wherein even low virus concentrations can be effective against a large volume of tumor mass through ongoing tumor-selective production of viral progeny. In experimental settings, using an MOI of <1 helps in assessing infectivity at a low dose, because viral replication is required to have a strong effect on a great number of tumor cells. Infection and cytopathic effects were monitored by phase-contrast microscopy for all tested viruses, and using fluorescent microscopy to detect viral GFP expression in all viruses except VSV-CT1 which does not contain a GFP reporter. Under control conditions, infection of U87 cells with VSV-rp30, VSV-M51, VSV-CT9, VSV-CT9-M51, and VSV-p1-GFP led to similar, widespread, nearly complete infection and the appearance of cytopathic effects, as with wild-type-based VSV-G/GFP, and only small differences were found between the variants. In contrast, replication-impaired VSV-dG-GFP and VSV-dG-RFP infected only a fraction of the cells in the culture dish. Interferon does not protect U87 cells from VSV infection. In the presence of IFN-α, infection and spread were slightly delayed, with little difference between VSV-G/GFP and VSV-rp30, VSV-M51, or VSV-CT9. However, the double mutant VSV-CT9-M51 and gene-shifted VSV-p1-GFP showed less cytopathic effect than VSV-G/GFP. Finally, low-dose VSV-dG-GFP and VSV-dG-RFP were strongly impaired in infecting U87 cells in the presence of IFN.

For quantitative assessment of cell viability, a colorimetric MIT assay was used to study the oncolytic action of 10 VSV variants on U87 cells and on normal human glia cells. To determine if IFN increases the selectivity of some of the viruses for cancer cells, cells were grown in 96-well dishes in the presence or absence of IFN-α (100 U/ml). To investigate which viruses performed well at a low virus concentration, virus was applied at an MOI of 0.5, and the MTT assay was performed at the indicated time points.

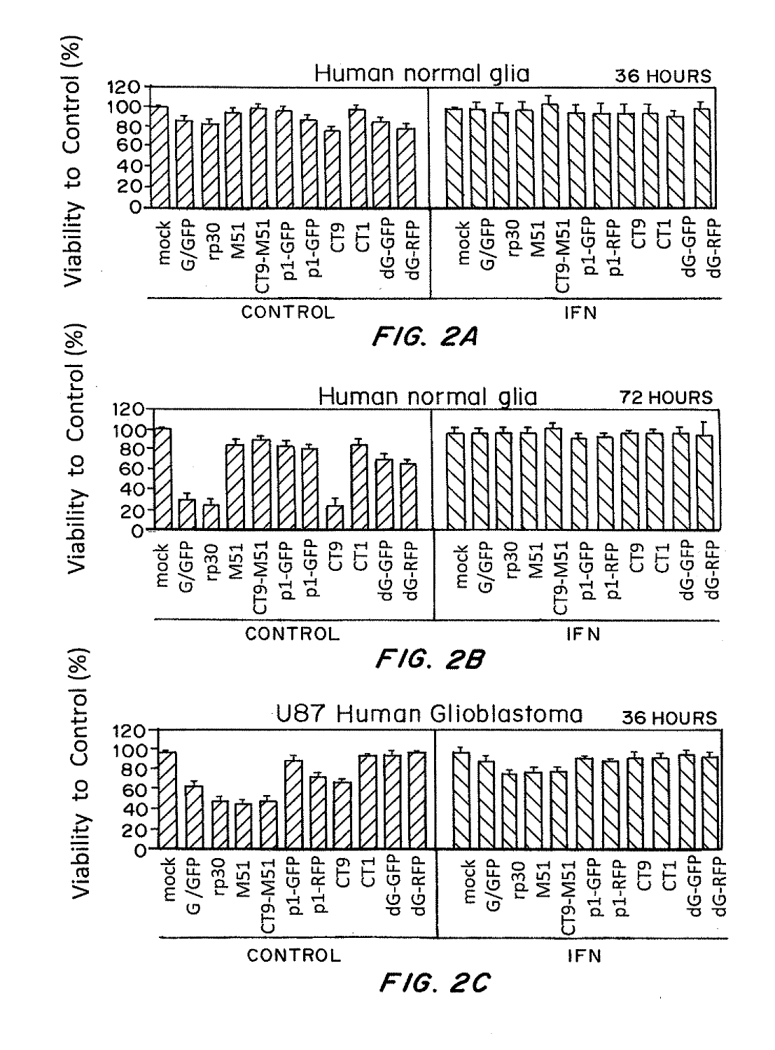

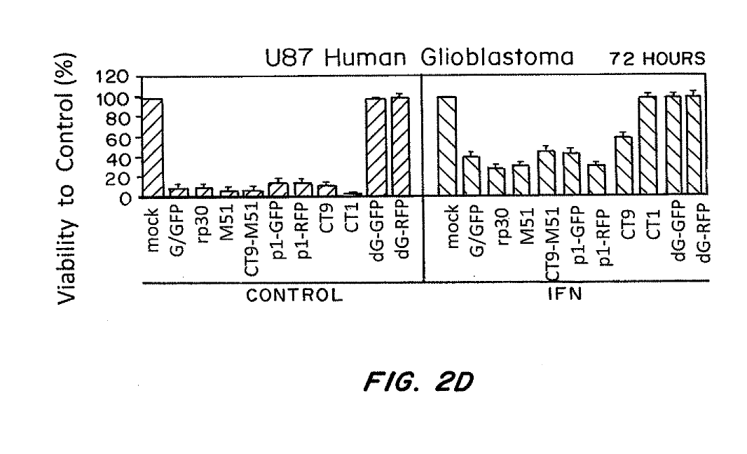

Thirty-six hours after inoculation of the 10 VSV variants, little effect on cell viability was seen in human glia control cells, with all viruses (except VSV-CT9 and VSV-dG-RFP) causing a <20% decrease in viability (FIG. 2 A). After 72 h, a significant decrease in cell viability was noted with VSV-G/GFP, VSV-rp30, and VSV-CT9. In contrast, cultures infected with VSV-M51, VSV-CT9-M51, VSV-p1-GFP, VSV-p1-RFP, and VSV-CT1 maintained viabilities of over 80% compared to mock-infected control cells (FIG. 2B).

On the other hand, complete protection from infection of any VSV variant was seen in control cells after preincubation with IFN-α, (right hand boxes in FIGS. 2A and 2B). Control cultures pretreated with IFN-α lacked any signs of GFP expression (data not shown), further supporting the protective role of IFN in controlling VSV infection in normal cells. In contrast, cell viability of U87 glioblastoma cells was significantly reduced, by 25 to 60%, compared to that of mock-treated control cells 36 h after infection with 6 of the 10 VSV variants, with VSV-rp30, VSV-M51, and VSV-CT9-M51 showing the most tumor cell killing (FIG. 2C). In the presence of IFN, VSV-rp30, VSV-M51, and VSV-CT9-M51 caused reductions of U87 cell viability of about 20%.

Underscoring the strong oncolytic potential of the viruses, tumor cell killing was nearly complete at 72 h postinfection (hpi), despite the low initial MOI of 0.5, in all but the two replication-restricted viruses, VSV-dG-GFP and VSV-dG-RFP (FIG. 2D). In addition, even after preincubation with IFN-α, 7 of the 10 VSV variants continued to infect and kill tumor cells, with a reduction in viability of 40 to 70%. The two replication-incompetent viruses, VSV-dG-GFP and VSV-dG-RFP, showed a poor ability to kill tumor cells in the presence of IFN and also showed only a modest effect under control conditions at a low MOI of 0.5; this was due in part to the inability of the viruses to generate second rounds of infectivity. VSV-CT1, which showed strong tumor cell killing under control conditions, was not effective at killing tumor cells in the presence of IFN. There is an apparent difference in that replication-restricted VSV-dG variants suppressed viability on human glia cell control cultures but not on human U87 glioblastoma cells. Since U87 tumor cells divide rapidly, the number of initially infected cells was outgrown by the dividing culture in 36 and 72 h (for VSV-dG-GFP and VSV-dG-RFP, respectively). In contrast, the proportion of infected glia control cells remained approximately the same in the course of the 3-day experiment.

Together, these initial in vitro experiments showed a number of VSV variants to be highly attenuated for control human glia cells but ineffective against U87 cells. These include the two replication-incompetent VSV-dG-GFP and VSV-dG-RFP variants and VSV-CT1. On the other hand, a number of VSVs are excellent in their antitumor action, but with noticeable toxicity on human control glia cells. These included VSV-G/GFP, VSV-rp30, and VSV-CT9. Finally, a third group emerged, with little toxicity against control cells yet reasonably good tumor cell killing, comprised of VSV-M51, VSV-CT9-M51, VSV-p1-GFP, and VSV-p1-RFP.

### Example 2

### Effect of VSV Attenuation on Viral Replication in Tumor and Control Cells

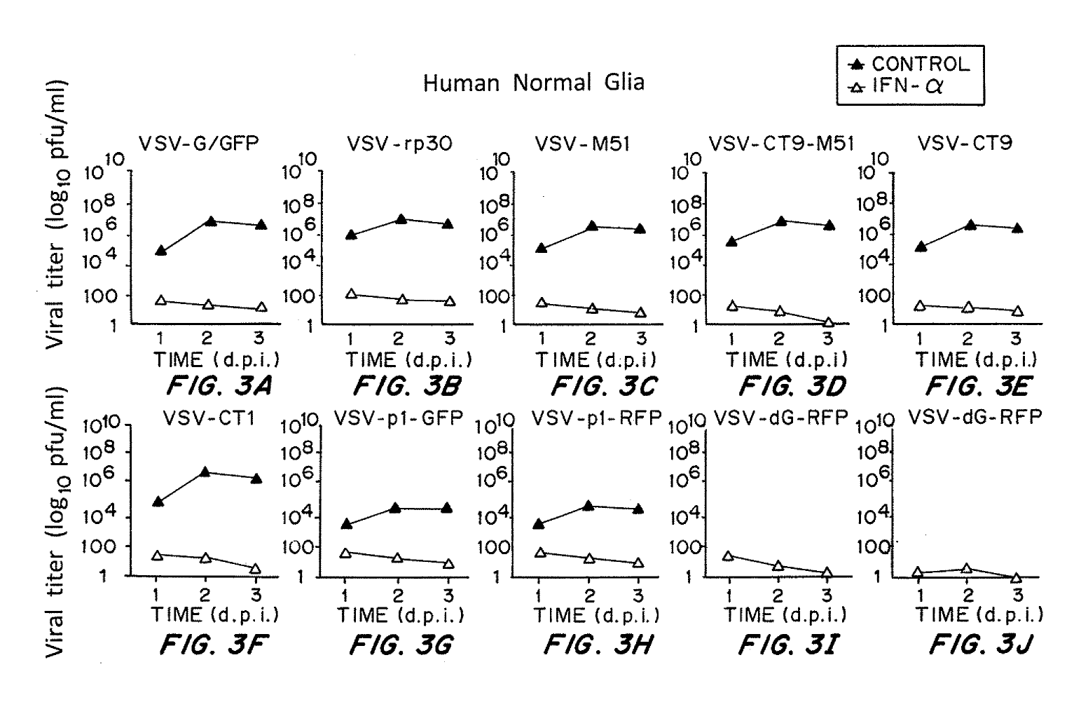

Local self-amplification is one of the mainstays of oncolytic virus therapy. Viruses selectively replicating faster in tumor cells than in normal cells would be expected to have a stronger oncolytic profile. A semiquantitative was used to measure relative viral replication in control versus glioblastoma cells, i.e., the ratio of replication. Standard plaque assay techniques were used to determine viral replication of the eight replication-competent VSV variants on U87 cells and compared it to replication on normal human control cells. Two replication-restricted VSV-dG variants were also included in the replication assay to provide a baseline value for noninternalized parent viral particles. Monolayers of each culture were infected with an MOI of 1 with the respective VSV variant, and cell culture supernatants were collected at 1, 2, and 3 days postinfection and frozen until further analysis. Experiments were again performed in the presence and absence of IFN-α. Of the 10 VSV variants tested, VSV-rp30, VSV-M51, VSV-CT9-M51, VSV-CT9, and VSV-CT1 all had similar growth curves on normal human glia cells to that of wild-type-based VSV-G/GFP, in contrast to VSV-p1-GFP and VSV-p1-RFP, which showed reduced replication, by ˜100-fold, and VSV-dG-GFP and VSV-dG-RFP, which, as expected, showed no replication (FIGS. 3A to 3J). In the absence of plaque formation for replication-restricted variant titers, VSV-dG variants were assessed by the number of individual infected cells expressing either the red or green fluorescence reporter gene. For all replication-competent VSV variants, viral replication was greatly reduced by IFN-α pretreatment.

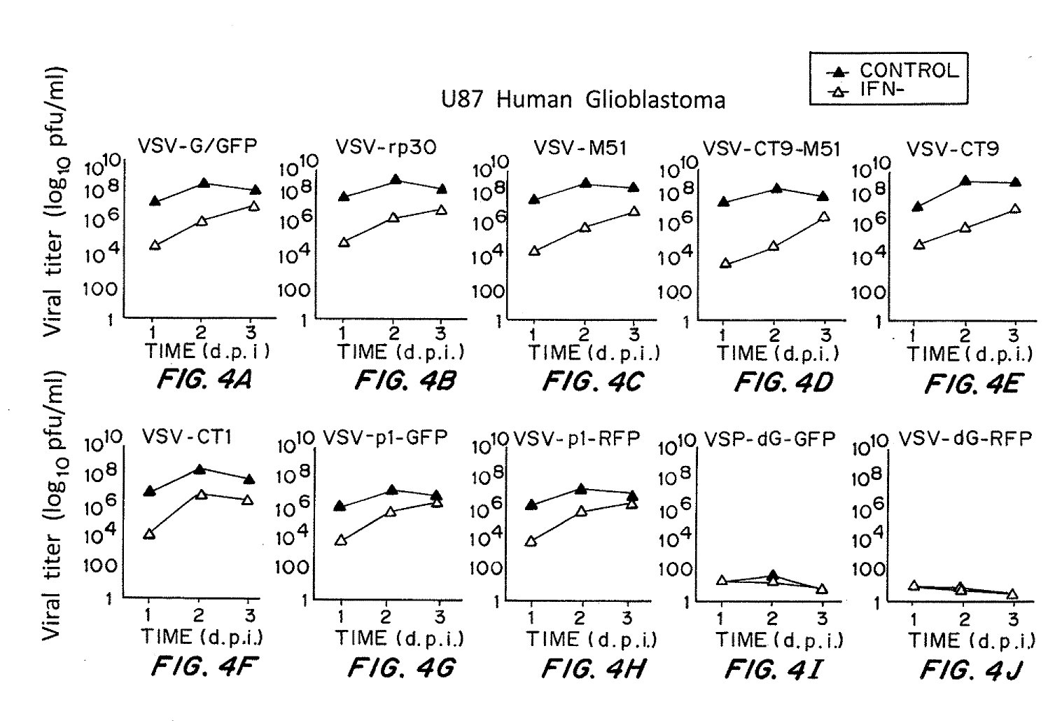

On U87 cells, viral replication was significantly higher (˜100-fold) than that on control cells for all but the two replication-deficient viruses, VSV-dG-GFP and VSV-dG-RFP. As on normal human glia cells, little difference was seen between VSV-rp30, VSV-M51, VSV-CT9-M51, VSV-CT9, VSV-CT1, and wild-type-based VSV-G/GFP (FIGS. 4A-4J). Calculating the maximum titer difference at 2 dpi for viruses under non-IFN control conditions between normal human glia cells and U87 cells resulted in the following ratios. These ratios are relevant and serve as an important index of the relative levels of VSV replication in normal and cancer cells. A large ratio is characteristic of a virus that shows substantially greater replication in cancer cells than in control cells. The ratios were as follows: VSV-G/GFP, 1:100; VSV-rp30, 1:121; VSV-M51, 1:287; VSV-CT9-M51, 1:341; VSV-CT9, 1:237; VSV-CT1, 1:74; VSV-p1-GFP, 1:386; and VSV-p1-RFP, 1:602. In contrast to the case with control human glia cells, interferon pretreatment did not prevent viral replication in U87 cells, with viral titers reaching similar values to those in non-IFN-treated controls by 3 dpi (FIGS. 4A-4J). The largest ratios were indicative of the most ideal viral candidates, namely, viruses that replicated more efficiently in cancer cells than in noncancer cells. The largest ratios were 1:386 and 1:602, for VSV-p1-GFP and VSV-p1-RFP, respectively. These contrasted with those of poor oncolytic performers, such as VSV-CT1, which had a ratio of 1:74 and was relatively ineffective at killing glioblastoma cells.

A primary mechanism of protection of normal cells against RNA viruses such as VSV is the activation of innate IFN pathways (Perry, et al., Cell Res., 5:407-422 (2005)). Several studies have indicate that many cancer cells have defective IFN response pathways (Stojdl, et al., Cancer Cell, 4:263-275 (2003)). Together, these findings show that IFN may selectively enhance the survival of normal cells over tumor cells in the presence of VSV. In the assays described above, IFN was effective at protecting normal cells from all VSV variants tested. However, in the presence of interferon, the oncolytic action against brain tumor cells was impaired with the strongly attenuated variants, VSV-CT1, VSV-dG-GFP, and VSV-dG-RFP. Three rVSVs (VSV-rp30, -CT9, and -G/GFP) showed more toxicity and greater replication on normal cells than did the other rVSVs. IFN completely reduced infection and replication in normal cells by all VSV variants. IFN has already been approved for use in the human CNS for treatment of multiple sclerosis (Goodin, Int. MS J., 12:96-108 (2005)), indicating that it has a strong safety margin within the brain. Thus, treatment of human brain tumors with recombinant VSVs may derive further benefit from coapplication of IFN in the brain to enhance the selectivity of the virus for the tumor, particularly with those viruses (VSV-G/GFP, VSV-rp30, and VSV-CT9) where infection of noncancer cells may be a problem. Although IFN may reduce infection by VSV, it did not greatly alter the ratio of infections in normal versus tumor cells for the top VSV candidates.

### Example 3

### Infection and Growth Suppression of Additional Human Glioma Cultures

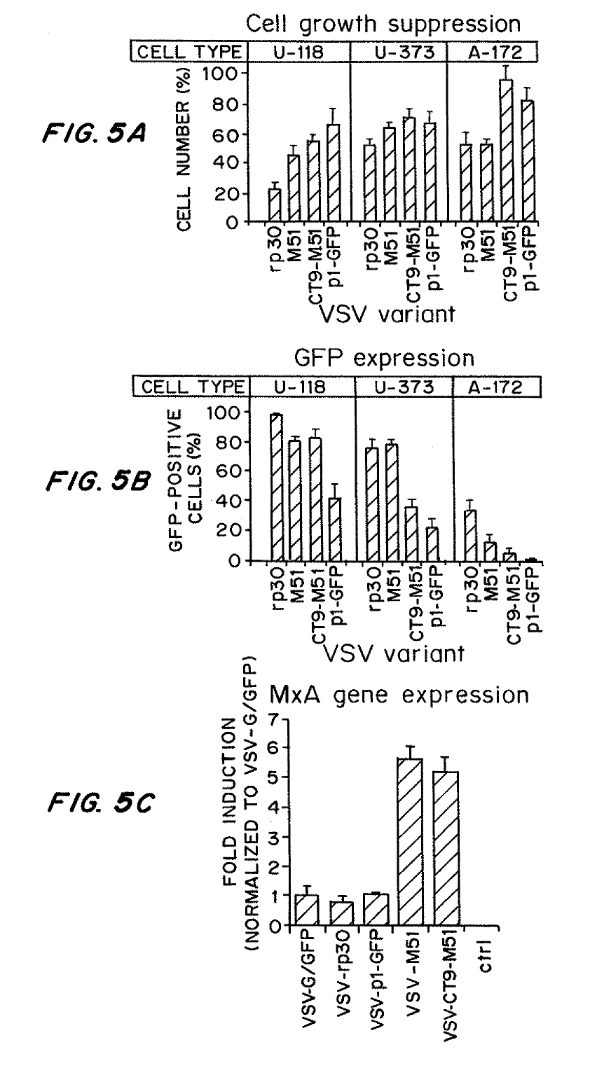

Glioblastoma tumors are characterized by heterogenous histology and mutation profiles. To test whether the effects of attenuated VSV mutants on U87 glioma infection and oncolysis can be generalized to other human glioblastoma cell lines, infections of three human cell lines were analyzed by the four most effective antitumor VSV variants, VSV-rp30, VSV-M51, VSV-CT9-M51, and VSV-p1-GFP. U118, U373, and A-172 cells were plated in 24-well dishes, infected at an MOI of 2, and analyzed 24 h later. Cell counting revealed cell growth suppression compared to noninfected controls for all VSV variants tested in all tumors (FIG. 5A). As in U87 cells, VSV-rp30 displayed the strongest suppression of tumor growth and cell lysis of up to 80% in U118 cells and 50% in both U373 and A172 cells. By 48 h, all cells were dead (data not shown). As seen with U87 cells, the other tested VSV variants displayed increasingly attenuated tumor suppression, in the order of VSV-M51, VSV-CT9-M51, and VSV-p1-GFR Using GFP fluorescence-reported infection, the infectivity of these VSV variants was monitored. VSV-rp30-infected cultures displayed the highest number of infected cells compared to VSV-p1-GFP, which showed the fewest cells infected (FIG. 5B). Together, these data mirror the trend that was seen with U87 glioblastoma cells. VSV-rp30 was found to be highly effective at targeting and killing glioblastoma cells, with the tested alternative VSV variants displaying an attenuated yet still effective antitumor profile.

In summary, viruses generated from a DNA plasmid substantively attenuated for virulence compared with wild-type VSV. Second, a transgene such as GFP or RFP to the viral genome helped in identifying infected cells but, importantly, also served to attenuate the resultant virus. This was particularly effective when the reporter gene was added at the first position, resulting in greater expression of the reporter gene than when it was placed in a secondary position and also causing a reduction in the expression of all five of the viral structural genes. VSV-CT9-M51, with a shortened cytoplasmic tail of the G protein and an M51 codon-deleted M gene, was further attenuated by a GFP reporter and by DNA derivation. The CT9 mutant by itself showed attenuated virulence, but interestingly, the combination of the CT9 mutation together with the M51 mutation gave a virus that behaved in a fashion roughly similar to that of virus with the M51 mutation alone.

### Example 4

### Differential Induction of Interferon Downstream Gene MxA

Materials and Methods

Quantitative Real-Time PCR

Normal human glia cells were grown in T25 flasks to confluence and infected with the respective VSV variants at an MOI of 2. After 6 h, RNA was extracted with TRIzol reagent (Invitrogen). Total RNA was reverse transcribed using a SuperScriptIII RT kit (Invitrogen) and random hexamer primers (Promega, Madison, Wis.). Primer selection and the PCR protocol have previously been described in detail (van den Poi, et al., J. Comp. Neurol., 516:456-481 (2009); Wollmann, et al., J. Virol., 81:1479-1491 (2007)).

Results

The innate cellular immune response plays a crucial role in controlling VSV infection in normal cells. MxA is a potent downstream gene of the activated interferon path. Significant differences in expression profiles of MxA after VSV-rp30 infection between five glioblastoma cell lines and a panel of three normal human glia cell cultures has been shown previously (Wollmann, et al., J. Virol., 81:1479-1491 (2007)). To address differences in the expressional response to different VSV variants, the induction of MxA was tested. A representative selection of different VSV mutants was used to infect triplicate cultures of normal human control glia cells at an MOI of 2. After 6 h, RNA was extracted and reverse transcribed. Quantitative real-time PCR revealed a five- to sixfold higher induction of MxA gene expression in cultures infected with VSV-M51 or VSV-CT9-M51 than in those infected with VSV-G/GFP, VSV-rp30, and VSV-1p-GFP (FIG. 5C), confirming the previously described ability of M51 mutants to increase the cellular interferon response due to the inability to block cellular gene expression (Stojdl, et al., Cancer Cell, 4:263-275 (2003)).

### Example 5

### Reduced Neurovirulence of Intranasally Applied VSV-p1-GFP

Materials and Methods

Animal Procedures

For intranasal application, young mice (p16) were mildly anesthetized with ketamine-xylazine and received 25 μl of virus solution in each nostril. The head was kept reclined and in a lateral position to enhance virus delivery to the roof of the nasal cavity. Mouse health and weight were monitored daily. Animals with either significant neurological symptoms (paralysis, lateropulsion, etc.) or a body weight drop below 75% of the starting value were euthanized according to institutional guidelines.

Results

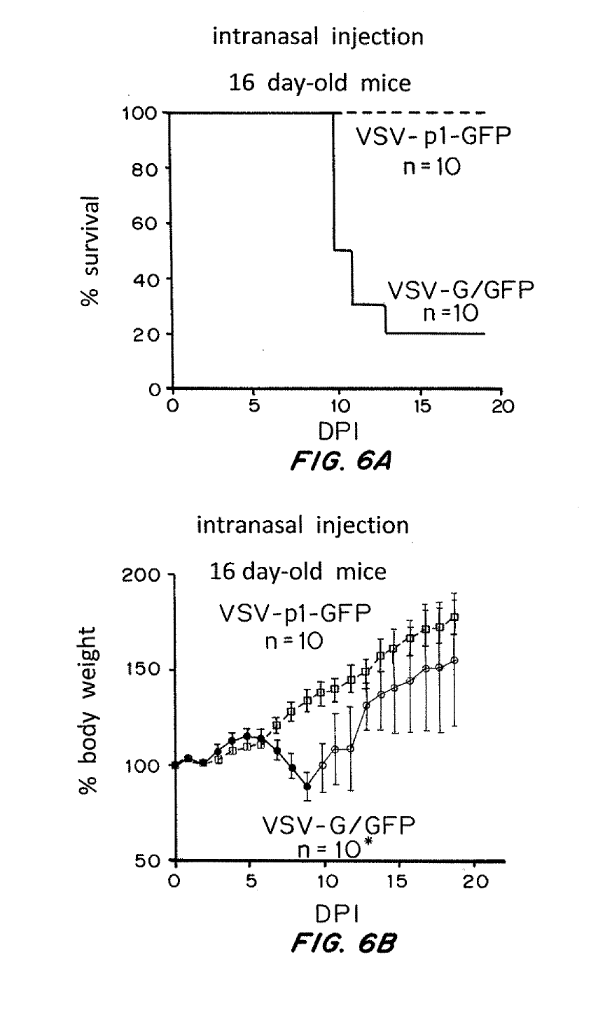

VSV may display neurovirulence in developing mice upon intranasal application (Lundh, et al., J. Neuropathol. Exp. Neurol., 47:497-506 (1988); van den Pol, et al., J. Virol., 76:1309-1327 (2002)). Based on the initial sets of in vitro experiments, the anti-tumor effects of an attenuated virus with a good antitumor profile, VSV-p1-GFP, was compare to wild-type-based VSV-G/GFP in a mouse model in vivo. Sixteen-day-old mice were given 250,000 PFU of either VSV-p1-GFP or VSV-G/GFP in each nostril, and mice were observed for neurological symptoms and weighed on a daily basis. FIG. 6A shows complete survival of 16-day-old mice (n=10) after VSV-p1-GFP application, compared to 80% lethality in VSV-G/GFP-treated mice (n=10). The corresponding body weight graph (FIG. 6B) displays a steady increase in weight in VSV-p1-GFP-treated mice and a significant drop in body weight in VSV-G/GFP-treated mice; the decrease in body weight was apparent after 5 dpi.

### Example 6

### Intravenous Application of VSV-p1-GFP Targets Intracranial Brain Tumor Xenografts

Materials and Methods

Animal Procedures

Four- to 6-week-old immunodeficient mice with a homozygous CB17-SCID background (CB17SC-M) (Taconic Inc.) were used for tumor xenograft experiments. A total of 1×10⁵ U87 glioblastoma cells expressing a red fluorescence reporter gene were injected stereotactically bilaterally into the striatum as previously described in detail (Oezduman, J. Neurosci., 28:1882-1893 (2008)). At 10 days postinjection, mice received a single bolus of 100 μl phosphate-buffered saline (PBS) containing 10⁷ PFU of VSV-p1-GFP in the tail vein. Animals were monitored with daily measurements of body weight, food and water consumption, and overall health. Two or 3 days later, animals were euthanized with a pentobarbital overdose and perfused transcardially with 4% paraformaldehyde. All animal experiments and postoperative care were performed in accordance with institutional guidelines of the Yale University Animal Care and Use Committee.

Results

Previous studies show VSV-rp30 (Oezduman, J. Neurosci., 28:1882-1893 (2008)) and VSV-M51 (Lun, et al., J. Natl. Cancer Inst., 98:1546-1557 (2006)) systemically target intracranial brain tumor xenografts after intravenous virus injection. Based on the initial in vitro experiments and the display of neuroattenuation after intranasal application of VSV-p1-GFP, the capability of this attenuated VSV variant to find and infect intracranial U87 xenografts after a single intravenous application was determined. It has been previously shown that peripheral inoculation with VSV does not target noncancer mouse or human control cells transplanted into the brain and does not target local brain injury at the same 10-day interval as that between cancer cell implantation and virus inoculation (Oezduman, et al., J. Neurosci., 28:1882-1893 (2008)). U87 cells that were stably transfected with monomeric RFP were used for tumor transplantation, allowing easy tracing and distinction from surrounding normal brain parenchyma. Human glioblastoma cells were injected bilaterally into the striatum of SCID mice. Ten days later, mice were given a single intravenous injection of 100 μl sterile PBS containing 5×10⁶ PFU of VSV-p1-GFP. Two mice each were sacrificed at 2 dpi and 3 dpi for histological analysis of virus infection of the tumor xenografts. All tumors were selectively infected with the virus, yet the surrounding brain appeared largely uninfected. All four animals bore sizeable tumors. Infection was monitored by fluorescent microscopy, where co-localization of GFP and RFP was indicative of infected tumor cells. All tumors were selectively infected with VSV-p1-GFP. A smaller tumor was completely infected at 3 dpi. Finally, VSV-p1-GFP infection was observed not only in the tumor bulk but also in small tumor islands dispersed around the main tumor.

The ability of VSV-p1-GFP to infect smaller tumor islands is important, as one of the chief clinical problems associated with glioblastoma is its tendency to migrate into normal brain tissue and thereby spread the cancer. Importantly, at the two time points analyzed, GFP expression was seen nearly exclusively in red fluorescent U87 cells, whereas the surrounding brain parenchyma was left largely uninfected. In a previous study (Oezduman, et al., J. Neurosci., 28:1882-1893 (2008), the activation of apoptosis in tumor cells infected with VSV-rp30 was confirmed using the same in vivo xenotransplant model. Morphological changes were similar to those described before. The tumors analyzed at 3 dpi showed cellular disintegration and blebbing of infected cells, which are typical of virally mediated oncolysis.

### Example 7

### Infectivity, Cytolysis, and Replication of VSV-G/GFP and VSV-rp30a in Human Sarcomas

Materials and Methods

Cell Culture

From the American Type Culture Collection (ATCC, Manassas, Va.), we obtained human fibrosarcoma HT-1080 (CCL-121), human osteosarcoma SJSA-1 (CRL-2098), human liposarcoma SW-872 (HTB-92), and baby hamster kidney cells (BHK). Normal human fibroblasts were purchased from Cambrex (Walkersville, Md.). Osteosarcoma Saos-2 cells were provided by Dan DiMaio (Yale University). Human synovial sarcoma SW982 (ATCC: HTB-93) and human bladder carcinoma T24 cells were provided by the Yale Cancer Center. Timothy Cripe (Children's Hospital Medical Center, Cincinnati, Ohio) generously provided human malignant fibrous histiosarcoma MFH-1, human osteosarcoma Osteomet/143.98.2 (CRL-11226), human rhabdomyosarcomas Rh30 and RD, malignant peripheral nerve sheath (MPNS) tumors S462-TY and STS-26T, and human Ewing's sarcoma family of tumors (ESFT) A637 and 5838 (Bharatan, N. S., et al. 2002. J. Pediatr. Hematol. Oncol. 24:447-453). Normal human decidual cells were a gift of F. Schatz and C. Lockwood (Yale University) (Lockwood, C. J., et al. 2011. Semin. Thromb. Hemost. 37:158-164). Primary mouse brain vascular endothelial (mBVE) cells were a gift of J. Madri (Yale University) (Li, Q., et al. 2011. Angiogenesis 14:173-185). Primary human glial cell cultures derived from patients undergoing epilepsy surgery were described previously (Wollmann, G., et al. 2010. J. Virol. 84:1563-1573).

Propagation

All cells were grown in Dulbecco's modified Eagle's medium (DMEM) with 10% fetal bovine serum (FBS), nonessential amino acids, 25 mM HEPES, and penicillin-streptomycin (pen-strep), except MFH1, Rh30, and 5838 cells, which were grown in RPMI with 10% FBS and pen-strep. Cells were grown at 37° C. with 5% CO₂.

Viral Preparations

VSV-G/GFP, a gift of J. Rose, expresses both the native G protein as well as a G/GFP fusion protein (between G and L genes) (van den Pol, A. N., et al. 2002. J. Virol. 76:1309-1327). VSV-rp30a, a derivative of VSV-G/GFP, was generated by positive selection and plaque purification on glioblastoma as previously described and contains two silent mutations and two missense mutations, one in P and one in L (Wollmann, G., et al. 2010. J. Virol. 84:1563-1573; Wollmann, G., et al. 2005. J. Virol. 79:6005-6022). Human cytomegalovirus (CMV) with a OFT reporter, from J. Vieira (University of Washington, Seattle, Wash.), was used as previously described (van den Poi, A. N., et al. 2007. J. Virol. 81:332-348). Sindbis virus expressing OFF was a gift of J. M. Hardwick (Johns Hopkins University, Baltimore, Md.) and used as previously reported (Wollmann, G., et al. 2005. J. Virol. 79:6005-6022).

Infectivity Assays

Cells (5×10⁴ per well) were seeded in 24-well dishes and incubated overnight. GFP reporter-expressing virus was added at the indicated multiplicity of infection (MOD. At the indicated times postinfection, the percentages of cells positive for GFP fluorescence were assayed by phase-contrast and fluorescence microscopy with an Olympus Optical (Tokyo, Japan) IX71 fluorescence microscope with a SPOT-RT camera (Diagnostic Instruments, Sterling Heights, Mich.). Each condition was assayed in triplicate wells. Within each well, total and GFP⁺ cells were counted in three adjacent fields using a 20× microscope objective. For longer-duration experiments in which cells were preincubated for 4 days with anti-IFN agents, 1×10⁴ cells were seeded instead.

Virus-Mediated Killing

Cells (5×10⁴ per well) were seeded in 24-well dishes and incubated overnight. Medium was changed to minimal essential medium (MEM) without phenol red, with 10% FBS and pen-strep, and VSV-G/GFP or VSV-rp30a was added at 5 PFU/cell. At 36 h postinfection (hpi), ethidium homodimer (EtHD) (Invitrogen, Carlsbad, Calif.) was added to a final concentration of 4 μM. After 30 min at 37° C., total cells and EtHD-positive (red fluorescent) cells were counted as for the infectivity assay.

Replication Assay

Cells (1×10⁵ per well) were seeded in 12-well dishes and incubated overnight. VSV-G/GFP or VSV-rp30a was added at 1 PFU/cell in 500 μl and incubated for 30 min at 37° C. Each well was washed 1 time with phosphate-buffered saline (PBS), and then 1 ml of growth medium was added. One-hundred-microliter samples of medium were taken at 24 hpi and stored at −80° C. until the time of analysis. Each experimental condition was replicated in triplicate wells, PFU/ml for each sample was determined by serial dilution and plaque assay on BHK cells, as previously described (van den Pol, A. N., et al. 2002. J. Virol. 76:1309-1327), performed in duplicate for each sample.

Statistical Analysis.

Comparisons were made by unpaired t tests, using KaleidaGraph software v3.6 (Synergy Software). Ratios were analyzed after logarithmic conversion of data [x′=log(x+1)] as recommended for statistical analysis of ratios (Ryder, E. F., et al. 2001. Curr. Protoc. Mol. Biol. Appendix 31).

### Results

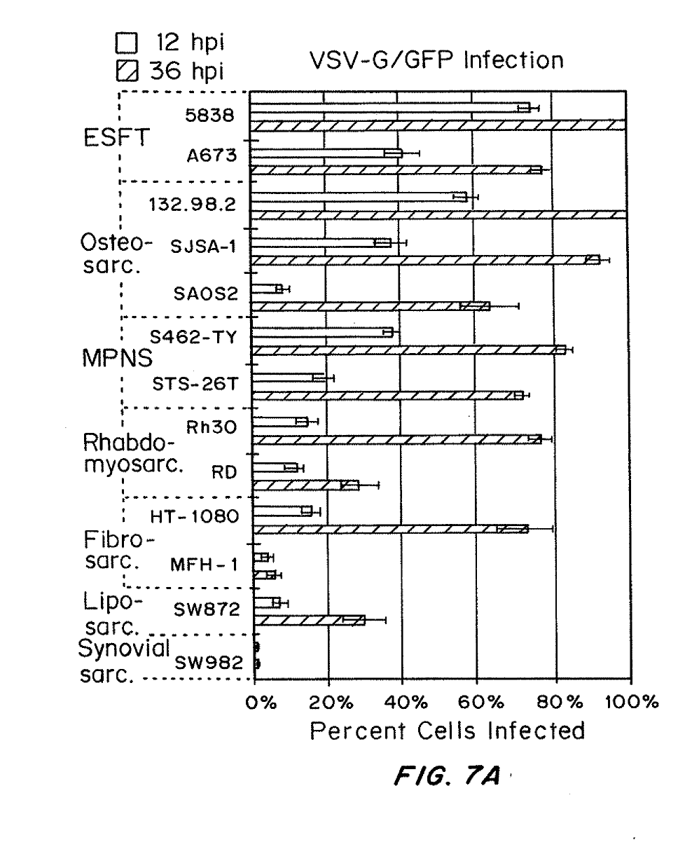

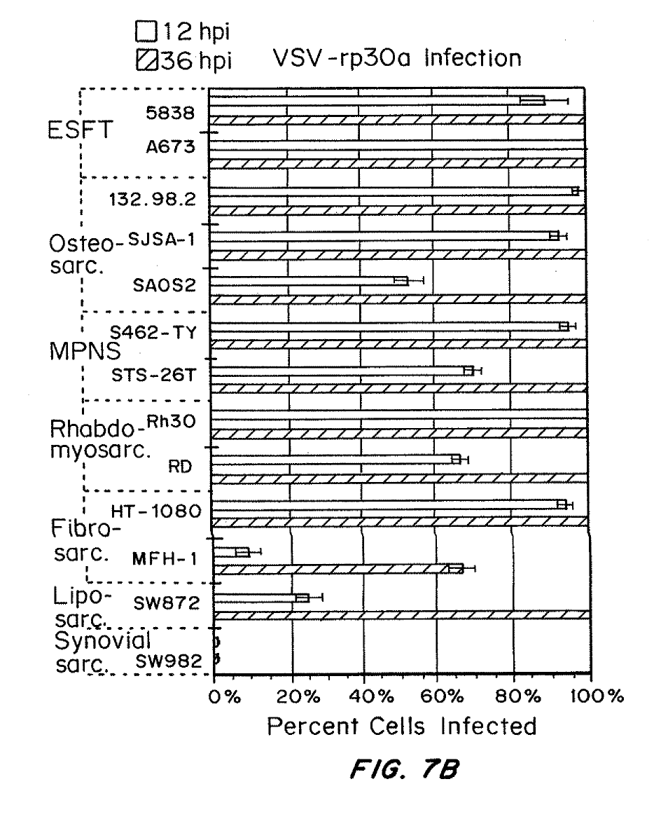

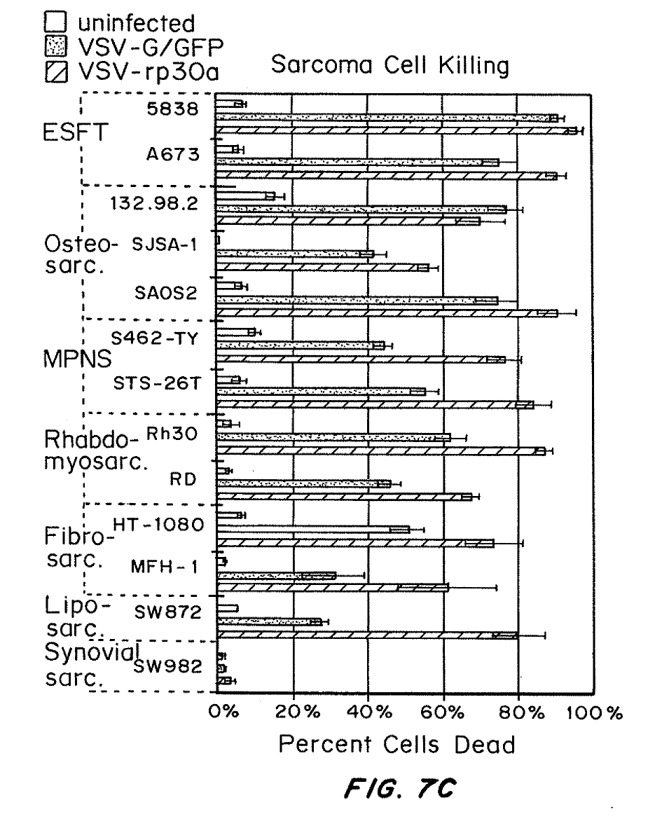

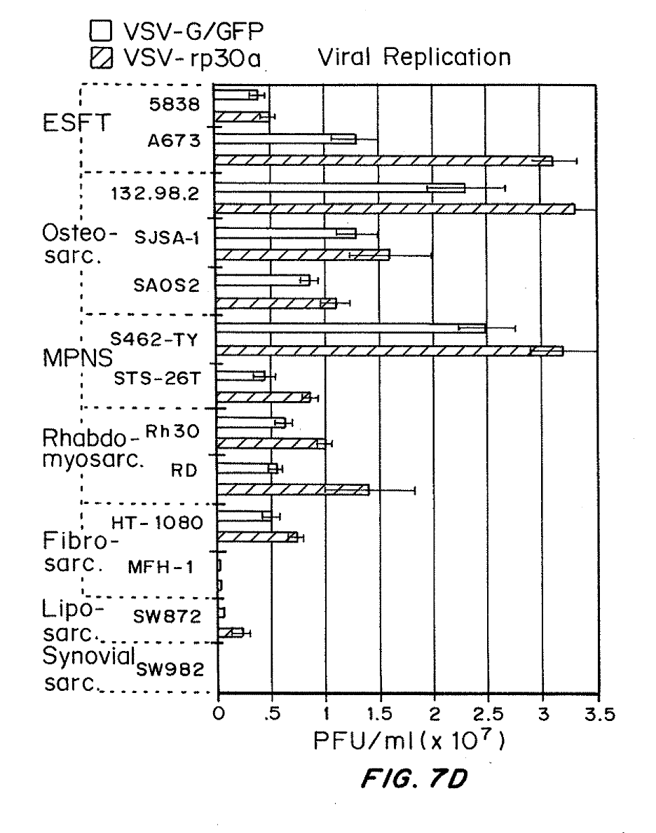

A panel of 13 human sarcoma lines was tested, representing seven sarcoma types: two Ewing's sarcoma family tumors (ESFTs), three osteosarcomas, two malignant peripheral nerve sheath (MPNS) tumors, two rhabdomyosarcomas, two fibrosarcomas, one liposarcoma, and one synovial sarcoma. All sarcomas were assessed after infection at an MOI of 5 with VSV-G/GFP or VSV-rp30a. Based on expression of the viral reporter gene, GFP, VSV-rp30a infected 100% of sarcoma cells within 36 hpi in 11 of the 13 sarcomas (FIG. 7B); in comparison, the infectivity of VSV-G/GFP was lower (FIG. 7A). At 12 hpi, the infectivity advantage of VSV-rp30a over VSV-G/GFP was universal (13 of 13 sarcomas) and averaged 3.6-fold (±0.53 standard error of the mean [SEM]) better across all sarcomas. There was no clear correlation between sarcoma tissue type and susceptibility to infection. The percentage of cells infected 12 hpi by VSV-rp30a averaged between 80% and 95% for all but three sarcoma lines (FIG. 7B, white bars), demonstrating that the high infectivity of VSV-rp30a generalizes to multiple sarcomas. Liposarcoma cells and fibrosarcoma MFH-1 cells were only moderately infected by VSV-rp30a at this time point, but by 36 hpi, all liposarcoma cells and the majority of MFH-1 expressed GFP, demonstrating successful infection. In contrast, synovial sarcoma SW982 was only 0.5% infected by VSV-rp30a at 12 hpi, and 0.8% infected at 36 hpi, demonstrating a unique and strong resistance to infection.

Ethidium homodimer (EtHD) was used as a label for dead cells. VSV-G/GFP-infected, VSV-rp30a-infected, and mock-infected cells were assessed 36 hpi (FIG. 7C). In 12 of 13 sarcoma cultures, infection with VSV-rp30a resulted in cell death for the majority of cells within 36 h. VSV-G/GFP also killed sarcoma cells in 12 out of 13 lines but with a rate that was statistically significantly lower than that for VSV-rp30a (P<0.05). For both viruses, the number of dead sarcoma cells at 36 hpi correlated well with the percentage of cells expressing GFP 36 hpi after infection with the same MOI (FIGS. 7A and B, black bars); EtHD-positive cells typically represented between 70 and 90% of infected GFP-expressing cells at this time point. No sarcoma cells were found that expressed the GFP reporter that did not eventually die. In contrast to these findings, only a small minority of infection-resistant synovial sarcoma SW982 cells were dead at 36 hpi. Only 1 to 2% of cells were EtHD positive, and this percentage was not different (P<0.3) from the percentage of cells dead (1%) in mock-infected cultures.

VSV progeny production was studied at 24 hpi (FIG. 7D). The three sarcomas with the highest amounts of viral replication were ESFT A673, osteosarcoma 132.98.2, and MPNS S462-TY, which were among those with high infection susceptibility (FIGS. 7A and B). The three sarcomas with the least replication efficiency were also those with the poorest infectivity: liposarcoma SW872, fibrosarcoma MFH-1, and synovial sarcoma SW982. VSV-rp30a showed a replication advantage (P<0.05) over VSV-G/GFP in 9 of 13 sarcomas and a trend toward advantage in 3 additional lines. This advantage averaged 2.0-fold (±0.26 SEM). In infection-resistant synovial sarcoma, progeny titers were so low (<250 PFU/ml) that they might represent only trace inoculum that remained on the cells despite washing, rather than true progeny. These titers are 800-fold (VSV-G/GFP) and 11.000-fold (VSV-rp30a) lower than those seen in MFH-1, otherwise the cell least supportive of VSV replication.

### Example 8

### VSV-rp30a is Selective for Sarcoma Over Normal Mesoderm

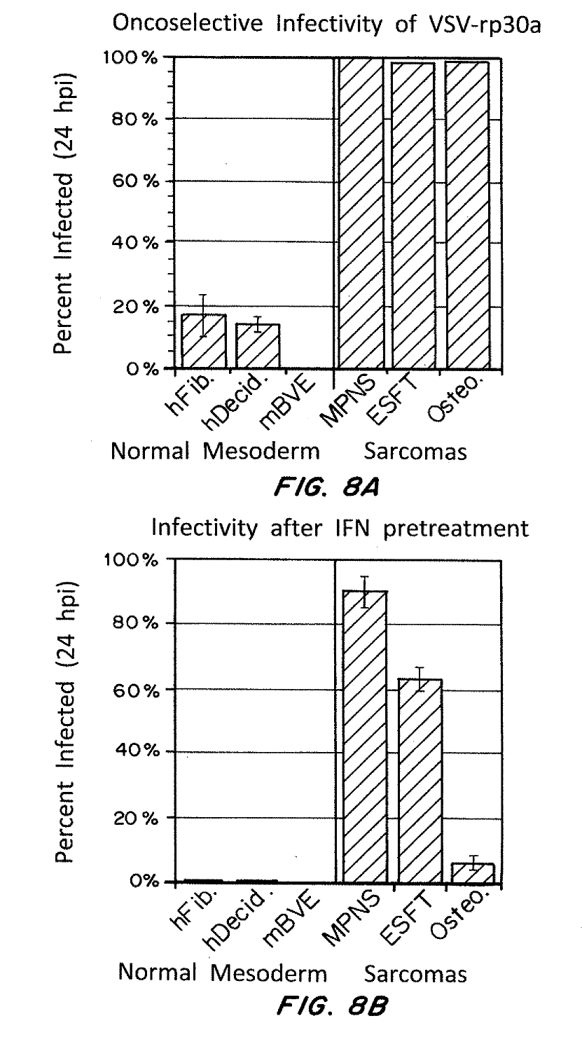

Cells or tissues with intact innate immunity can limit the number of rounds of replication that VSV can successfully achieve. To assess the oncoselectivity of VSV in normal versus sarcoma mesoderm, a panel of three normal mesoderm derived cells and three sarcomas were infected. Normal primary human fibroblasts, primary human decidua, and primary mouse brain vascular endothelium (mBVE) cultures, as well as three human sarcoma lines, were infected with VSV-rp30a (0.2 PFU/cell), and the percentage of cells infected was assessed 20 hpi (FIG. 8A). Infection rates in the three sarcomas averaged 9.9-fold more than in the three primary cell cultures, demonstrating oncoselectivity. mBVE cells were particularly resistant (<0.1% infected), an important finding in light of the fact that vascular endothelium is exposed to high virus titers during intravenous administration, as in our in vivo model below.

Within normal tissues and within tumor tissues, interferon secretion may be initiated by virus infection and may protect nearby cells. The infectivity of VSV-rp30a (MOI 0.2 PFU/cell) was therefore tested against a panel of normal and transformed cells after preincubation for 6 h with 100 units/ml of universal IFN-α (FIG. 8B). Normal primary cells were all VSV resistant after IFN pretreatment, with fewer than 1% of the cells expressing GFP at 20 hpi. Sarcomas were all infected but to different degrees. MPNS tumor S462-TY (100% infected in the absence of IFN) was 90% infected, representing only a small degree of IFN-mediated protection. ESFT A673, 98.8% infected in the absence of IFN, was 62.9% infected. Osteosarcoma 132.98.2, 99.1% infected in the absence of IFN, was only 5.8% infected, indicating a high degree of IFN protection in early stages of infection. Thus, sarcomas are highly variable in the degree to which they can be protected from infection by exogenous IFN.

All cultures were observed for additional time. Unprotected sarcoma cells were 100% infected and lysed by 2 days postinfection (dpi). IFN-protected sarcoma cells were 100% infected and lysed by 3 dpi, and cell death was confirmed by EtHD staining. In contrast, a significant portion of each of the three unprotected normal mesodermal cultures remained uninfected 3 dpi, and IFN-protected cultures were all less than 1% infected. The percentages of unprotected human fibroblasts, human decidua, and mBVE cells infected 3 dpi were approximately 30%, 50%, and 1%, respectively. Overall, these data are evidence that over time, sarcomas are unable to evade complete destruction after low MOI infection by VSV, even when protected in advance with IFN, whereas normal mesodermal cells in culture are VSV resistant to varying degrees without IFN pretreatment and are nearly 100% protected by IFN pretreatment.

### Example 9

### VSV-rp30a Selectively Infects Subcutaneous Sarcoma Xenografts and Arrests Tumor Growth In Vivo

Materials and Methods

Mouse Procedures

Animal experiments were approved by and performed in accordance with institutional guidelines of the Yale University Animal Care and Use Committee. Immunodeficient homozygous CB 17-SCID mice 7 to 8 weeks of age were obtained from Taconic Farms (Germantown, N.Y.). Bilateral subcutaneous flank tumors were established by injection of 5×10⁵ sarcoma cells in 100 μl of PBS. One dose of 5×10⁷ PFU of VSV-rp30a in 100 μl was injected via tail vein when tumor volume averaged approximately 120 mm3. Tumor dimensions were measured by caliper, and tumor volume (V) was estimated by the formula for the volume of an ellipsoid of length a and uniform width b, i.e., V=4/3π(a/2)(b/2)(b/2). For histology, animals were killed with a pentobarbital overdose and perfused transcardially with 4% paraformaldehyde in PBS.

Results

To assess the ability of systemic VSV-rp30a to specifically infect sarcoma in vivo, bilateral subcutaneous implantations of fibrosarcoma HT1080 and ESFT A673 were performed in SCID mice. When tumors averaged approximately 120 mm³, 5×10⁷ PFU of VSV-rp30a was injected via tail vein. Mice bearing HT1080 and A673 tumors were euthanized 6 or 7 dpi. Of nine established fibrosarcoma tumors, VSV-rp30 infection was found in all nine. Infection was generally widespread within the tumor, infecting approximately 90% of the cells. Adjacent normal tissue was consistently uninfected. Similarly, liver and spleen tissue showed no sign of infection at the same time point. GFP labeling of cell membranes, as shown, is characteristic of G/GFP fusion protein expression. Regions of the tumor with only weak fluorescence at low magnification often were seen to display this infection-specific pattern of GFP expression as well, suggesting these areas are at an early stage of infection. Of 11 established A673 tumors, all 11 were infected, although to a lesser degree than HT1080 tumors at the same time post-infection. The observed selectivity of VSV for sarcomas in this model is significant because VSV is not a species-restricted virus and can infect both mouse and human cells.

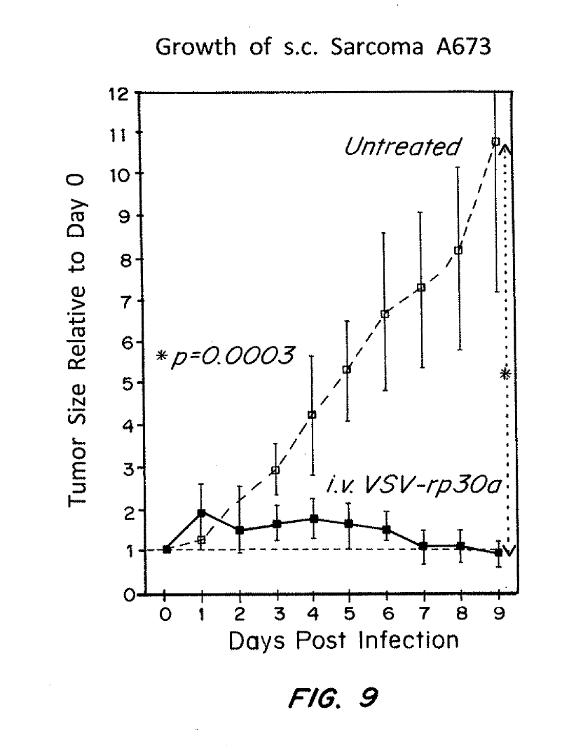

To assess the ability of intravenous VSV-rp30a to affect the growth potential of infected sarcoma xenografts, bilateral subcutaneous A673 tumors were established in 10 SCID mice. Successful tumor development in the majority of implantations, combined with injection of VSV-rp30a in five mice on day 13 after sarcoma implantation, was followed by daily measurement of tumor volume for 8 virus-treated tumors versus 6 untreated tumors. Mean volume of these tumors on the day of injection was 128 mm³ for untreated mice and, similarly, 116 mm³ for treated mice (non-significant, P>0.05). Mean changes in tumor volume (each tumor compared to its own volume on the day of injection) for 9 days following treatment are shown in FIG. 9 for treated versus untreated tumors. Untreated A673 tumor size increased by 10.8-fold, in significant contrast (P=0.0003) to treated tumors, which did not grow, and further had a trend toward decreasing size (90% of original volume). This experiment demonstrates the ability of intravenous VSV-rp30a to find, selectively infect, and arrest the growth of an otherwise rapidly growing sarcoma in vivo.

The studies above show that the vast majority of a representative panel of human sarcoma cell lines are highly susceptible to VSV-induced oncolysis, especially to VSV-rp30. In the following, we address the mechanisms underlying the resistance of synovial sarcoma to VSV.

### Example 10

### VSV-Resistant Synovial Sarcoma SW982 are Permissive for Viral Binding and Endocytosis

Materials and Methods

Cell Association Assay

Cell association of VSV-rp30a was measured under four different conditions as described (Ozduman, K., et al. 2008. J. Neurosci. 28:1882-1893). In all cases, 12-well dishes were seeded with 200,000 cells per well, exposed in quadruplicate to 10 PFU/cell of VSV-rp30a under the conditions specified, and washed five times with PBS plus 5 mM Ca²⁺ before RNA harvest and genome titration by quantitative RT-PCR (qRT-PCR) (see below). To assess membrane binding under conditions that inhibit endocytosis, virus and cells were incubated for 20 min at 4° C. (“binding”). To assess binding and endocytosis together, infected cells were incubated 30 min at 37° C. (“30 min”). To measure kinetics of early cell cycle events beyond 30 min, cells were washed after a 30-min incubation at 37° C. and incubated an additional hour at 37° C. (“90 min”). Finally, to assess cell association under conditions that block endosomal acidification and therefore viral escape from the endosome, the 90-min conditions were replicated but in the presence of 5 mM ammonium chloride (“endosomal”). Cells were washed 5 times, total RNA was harvested, and VSV-genome concentration was measured by quantitative RT-PCR.

Results

The paucity of GFP expression among VSV-infected synovial sarcoma cells suggested a significant block to an early step in the viral life cycle, perhaps binding or cell entry. This possibility was tested by measuring the efficiency of cell association of VSV-rp30a under four different conditions. Synovial sarcoma was compared to highly VSV-susceptible sarcoma S462-TY, after addition of VSV-rp30a at 10 PFU/cell.

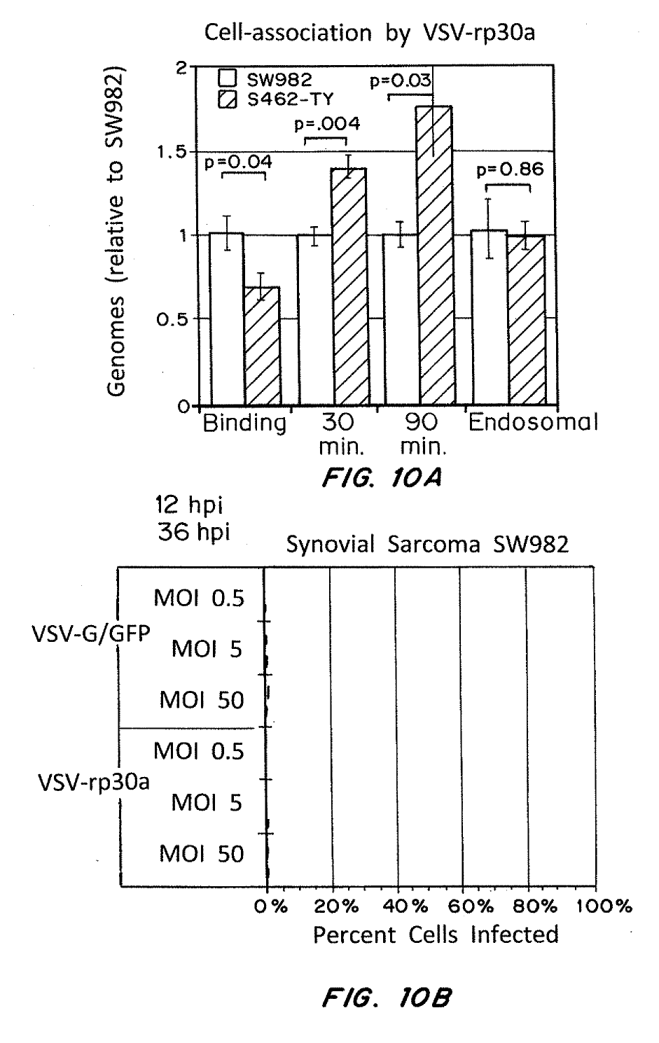

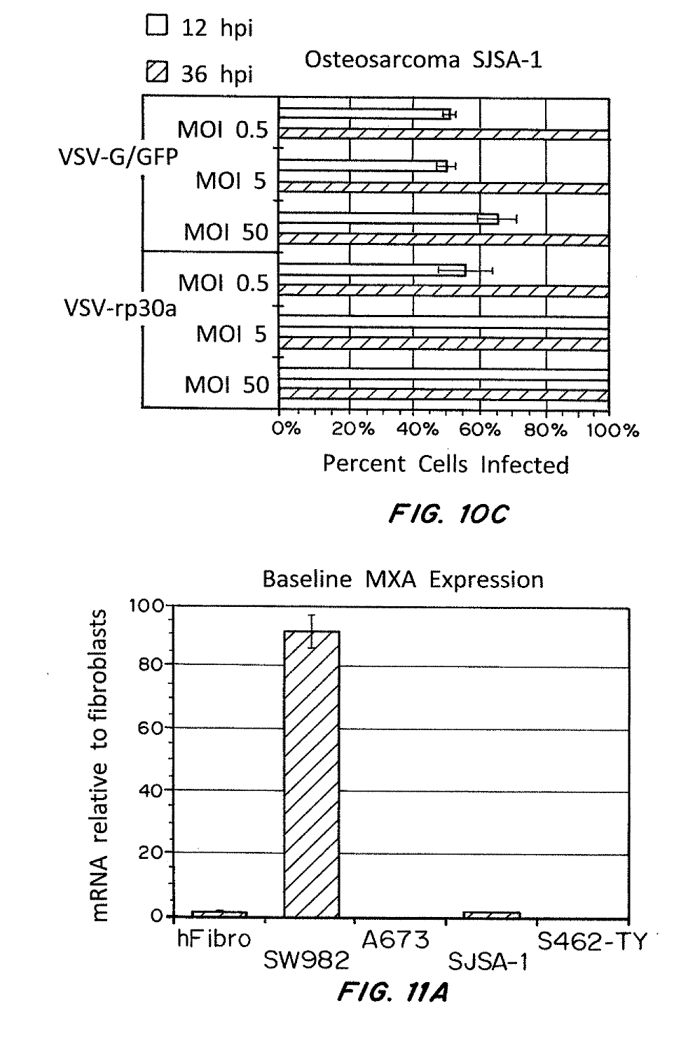

Binding of VSV-rp30a to synovial sarcoma SW982 was slightly more efficient than binding to MPNS tumor S462-TY (1.5-fold), indicating that lack of viral receptor does not explain the resistance of SW982. Under the 30-min and 90-min conditions, there were slightly more genomes associated with MPNS than with synovial sarcoma (1.4-fold and 1.8-fold, respectively). However, under the set of experimental conditions in which viral escape from the endosome is blocked (“endosomal”), genomes in infected synovial sarcoma and MPNS were equivalent (FIG. 10A). Taken together, these data indicate that VSV-resistant synovial sarcoma SW982 cells are not substantively deficient for VSV binding or endocytosis relative to a highly susceptible sarcoma.

### Example 11

### Synovial Sarcoma SW982 is Profoundly Resistant to Hightiter VSV, Sindhis, and Cytomegalovirus

Among all the sarcomas tested, the resistance to VSV observed in synovial sarcoma SW982 stood apart as unique. Even when the MOI was raised to a very high level of 50 PFU/cell, VSV-G/GFP and VSV-rp30a still infected only a small fraction of cells, less than 1% for each virus, at both 12 and 36 hpi (FIG. 10B). At each of these MOIs, by 36 hpi, VSV-G/GFP and VSVrp30a both infected 100% of other sarcoma cells—osteosarcoma SJSA-1 infected in parallel (FIG. 10C).

To test the hypothesis that synovial sarcoma has a high resistance to viral infection in general, the VSV-resistant synovial sarcoma was infecting with other unrelated viruses. To ensure that these viruses were capable of infection, three other sarcomas that were susceptible to VSV (SJSA-1, A673, and S462-TY) were also tested. GFP-expressing human cytomegalovirus, a double-strand DNA virus in the family Herpesviridae, and Sindbis virus, a positive-RNA-strand alpha virus in the family Togaviridae, were used. The synovial sarcoma was highly resistant to both Sindbis and CMV; only a small minority (<1%) of cells showed signs of infection at 24 and 48 hpi. In contrast, CMV and Sindbis efficiently infected other sarcoma cells tested, the majority of cells being GFP positive by 48 hpi. ESFT A673 showed a level of resistance to CMV comparable to that seen in synovial SW982, despite the susceptibility of these cells to VSV and Sindbis. Together, these results indicate a general resistance of this synovial sarcoma to multiple types of viruses unrelated to VSV.

### Example 12

### VSV-Resistant Synovial Sarcoma SW982 has High Basal Expression of Interferon-Stimulated Genes

Materials and Methods

Quantitative RT-PCR

RNA was extracted from cell lysates by using TRIzol (Invitrogen, Carlsbad, Calif.) and reverse transcribed by random hexamer priming by using the Super Script III reverse transcriptase kit (Invitrogen). For host cell gene expression analysis, TaqMan gene expression assays (Applied Biosystems, Foster City, Calif.) for hIFN-β, ISG-15, MxA, OAS-1, ISG-56, and β-actin were acquired and used as recommended by the manufacturer. For measurement of VSV genomes, a TaqMan assay was used in which primers span the junction between N and P genes; gene spanning reduces detection of mRNA transcripts, which are monocistronic. Bach sample was measured in triplicate PCRs, and the result was internally normalized to β-actin expression levels in that sample, also measured in triplicate.

Results

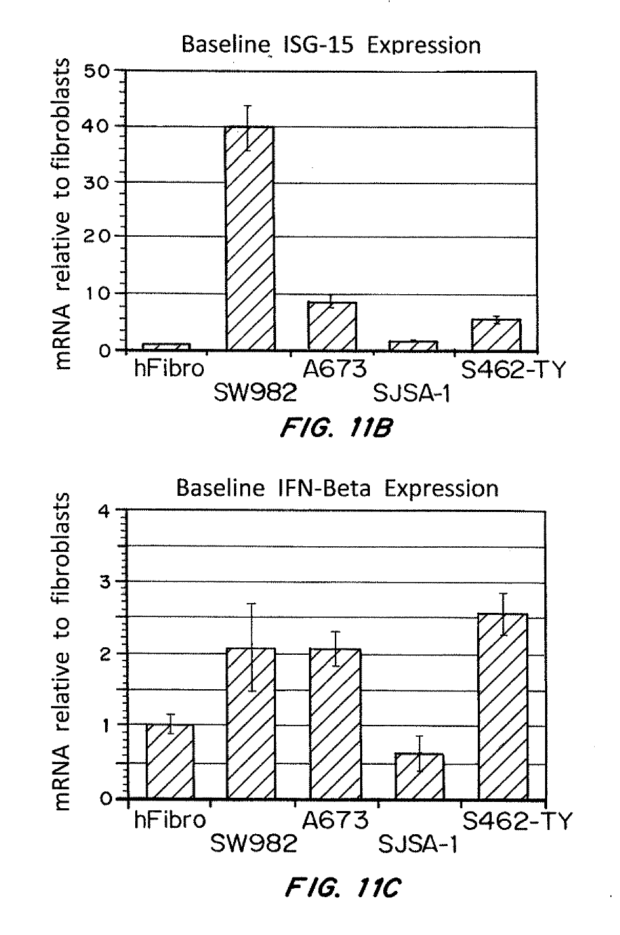

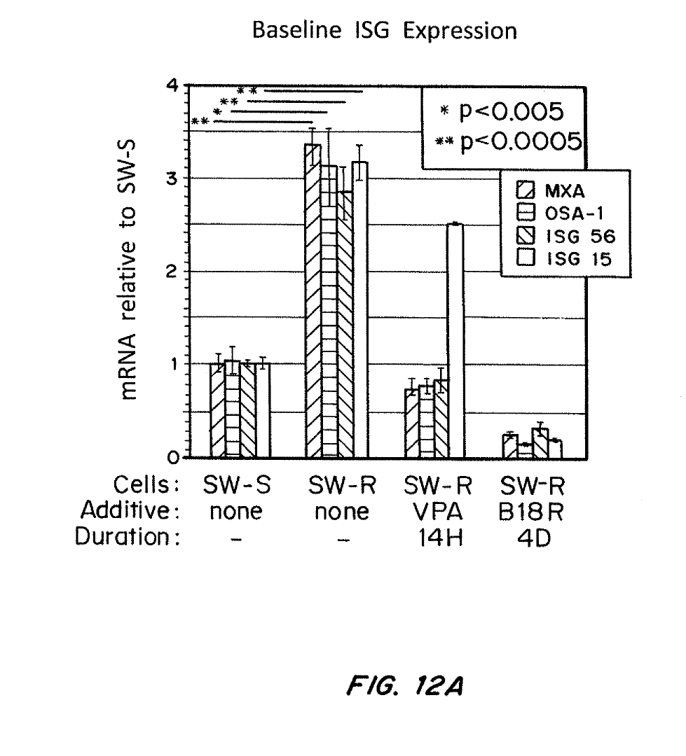

Given the uniform resistance of SW982 to highly divergent viruses, and verification that binding and endocytosis were intact, it was postulated that innate immunity may be responsible for this unique degree of resistance. One hypothesis is that VSV infection of these cells induces a stronger upregulation of IFN-β and downstream interferon-stimulated gene (ISG) expression in response to VSV, compared to susceptible cells. However, in measuring the mRNA levels of IFN-β, ISG-15, and MxA 6 hpi in VSV-infected versus mock-infected synovial sarcoma, and in fibroblasts, a greater relative postinfection upregulation of all three genes was observed in normal fibroblasts compared to the relatively resistant SW982. Surprisingly, the basal, i.e., preinfection, expression levels of ISGs MxA and ISG-15 were both remarkably higher in SW982 than in normal cells (90-fold and 36-fold, respectively) (FIGS. 11A and 11B).

This finding led to the hypothesis that relative to various susceptible sarcomas, SW982 might be unique in having aberrantly high expression of ISGs prior to infection, and that this may account for the resistance phenotype. Basal expression of MxA and 1SG-15 was measured in three VSV-susceptible sarcomas (A673, SJSA-1, and S462-TY). Uninfected SW982 expressed both of these ISGs at much greater levels than did uninfected VSV-susceptible sarcomas: on average, synovial sarcoma cells showed a 270-fold higher expression for MxA (P=0.002) and 12-fold higher expression for ISG-15 (P=0.03) (FIGS. 11A and 11B).

If ISGs were globally upregulated in SW982 in advance of infection, the direct antiviral activities of several of these proteins might abrogate the viral life cycle very early in infection, thus preventing GFP reporter expression. The MxA protein, for example, blocks rhabdoviruses early in the life cycle by binding nucleocapsids (Sadler, A. J., et al. 2008. Nat. Rev. Immunol. 8:559-568). The expression of ISGs is regulated by JAK/STAT signaling downstream of receptors for IFN (Randall, R. E., et al. 2008. J. Gen. Virol. 89:1-47), raising the possibility that SW982 cells secrete increased IFN constitutively. Using specific primers for IFN-β, elevated basal expression levels were not detected in SW982 cells relative to the average expression in other cells tested (P=0.33) (FIG. 11C). The possibility remained that SW982 could be secreting another IFN, such as one of the multiple alpha interferons, or that ISGs were upregulated by an IFN-independent mechanism.

The ability of medium from cultured cells to inhibit VSV infection is a commonly employed bioassay for interferon (Meager, A. 2002. J. Immunol. Methods 261:21-36). However, when we conditioned medium on synovial sarcoma overnight and exposed human fibroblasts to this medium for 4 h, we observed no effect on the percentage of cells infected 24 h postinfection with VSV-G/GFP (1 PFU/cell), as compared to fibroblasts infected after exposure to fresh medium or medium conditioned on fibroblasts, despite the ability of IFN-α-spiked medium (200 U/ml) to completely protect these cells from VSV after the same 4-h incubation period. This result suggested that SW982 cells do not show enhanced interferon secretion at a level sufficient to protect other cells from VSV.

### Example 13

### Rare VSV-Susceptible Cells within SW982 can be Grown into Single-Cell Subclones with Significant and Stable VSV Susceptibility

### Materials and Methods

### Isolation of subclones of synovial sarcoma

Single-cell-derived subclone populations of SW982, i.e., SW-S and SW-R, were isolated in individual wells of a 96-well plate. SW982 cells were diluted to 0.5 cells per 100 μl in medium that was 50% conditioned, and 100 μl was seeded per well. Wells with individual colonies were propagated and cultures of the desired virus-resistant and virus-susceptible phenotypes were identified.

Results

A small minority (<1%) of the SW982 population were VSV susceptible, expressing GFP at high levels despite the absence of GFP in the great majority (>99%) of the culture. This heterogeneity of phenotype raised the question of whether the susceptible subpopulation might harbor a genetic anomaly. Intratumoral diversity at the genetic level often results in variability in cell phenotype within the population, including variability in susceptibility to tumor killing (Gerlinger, M., et al. 2010. Br. J. Cancer 103:1139-1143). A strategy was adopted that involved growing single-cell-derived subclones of tumor cell lines to study this diversity (Held, M. A., et al. 2010. Cancer Res. 70:388-397). Two phenotype-homogenous cell lines were expanded. The SW-S subclone was highly susceptible to VSV. In contrast, the SW-R was very resistant to infection; even with very high MOIs (50 and 100 MOD of VSV, no infected cells were found.

Cytogenetic analysis of clonal synovial sarcoma lines (and the parental line, SW-P) was performed to verify their identity as synovial sarcoma rather than a contaminating cell line, often an unrecognized problem in cultured cells (Lacroix, M. 2008. Int. J. Cancer 122:1-4) and to determine if there were any unique cytogenetic features. Six metaphases each of SW-P, SW-R, and SW-S were analyzed. All three lines shared multiple karyotypic alterations, specifically 47, XX, t(1; 4; 9) (q12; q11,p24), del(S) (q31q33), der(9,13) (q10q10), +der(20) t(5;20) (q11.2:p13), +mar[6]. Rouleau et al. (35) reported nearly identical karyotype features for SW982: 47-49, XX, t(1; 4; 9) (q12; q11; p24), del(5) (q22q33), der(9; 13) (q10; q10), +der(20) t(5; 20) (q11.1; p13). The karyotype analysis supports the view that the susceptible subpopulation, SW-S, is not a contaminating cell line but instead is closely related to the parent SW982 cells. Additionally, SW-S had three unique features, namely, the addition of unidentified chromosomal material to a long arm of chromosomes 7, 11, and 18, any of which might be related to its enhanced susceptibility, although a cytogenetically undetectable mutation may also be the causative mutation specific to these cells.

### Example 14

### Preadministration, but not Coadministration, of Agents that Antagonize Interferon Signaling Renders VSV-Resistant Sarcoma Cells Susceptible

Materials and Methods

Exogenous Agents

Valproate (VPA) and universal IFN-αA/D were purchased from Sigma (St. Louis, Mo.) and Jak inhibitor I was from Calbiochem/EMD Chemicals (Gibbstown, N.J.). Vaccinia virus B18R protein preparation was generously provided by Michael Robek (Yale University) (Sandi, P., et al. 2010. J. Interferon Cytokine Res. 30:123-134). B18R was used at a concentration sufficient to neutralize 10 ng of type I IFN per milliliter.

Results

In the resistant synovial sarcoma cells, elevated basal ISG expression was found rather than a robust ISG response to infection. If an upregulated ISG system is the mechanism responsible for the remarkable resistance of synovial sarcoma cells to VSV, then reducing the baseline ISG expression should increase the level of infection in resistant cells. Three converging approaches were therefore tested to interfering with ISG expression. These included disrupting gene regulation with histone deacetylase inhibitors, disrupting second messenger signaling after IFN binds to its receptor with a JAK1 inhibitor, and reducing IFN binding to type I IFN receptors with poxvirus B18R protein.

Histone deacetylase inhibitors such as valproic acid (VPA) disrupt gene expression and can impact cellular resistance to viral infection (Diallo, J. S., et al. 2010. Mol. Ther. 18:1123-1129; Nguyen, T. L., et al. 2008. Proc. Natl. Acad. Sci. U.S.A. 105:14981-14986; Otsuki, A., et al. 2008. Mol. Ther. 16:1546-1555). VPA (30 mM) was added to the medium of SW-R cells at different times relative to infection. Neither cotreatment nor 14-h pretreatment increased susceptibility, despite the finding that a 14-h preincubation with VPA reduced basal mRNA levels for three out of four ISGs in SW-R to levels comparable to those in SW-S (FIG. 12A). As a longer preincubation may be necessary to fully reduce ISG protein levels, a 4-day preincubation was used. This 4-day VPA treatment resulted in a dramatic reversal of the VSV-resistant phenotype of SW-R and greatly enhanced infection.

Signaling downstream of the type I IFN receptor and resultant ISG expression are dependent on tyrosine kinase JAK1 (Randall, R. E., et al. 2008. J. Gen. Virol. 89:1-47). As an additional test of the hypothesis, SW-R was exposed to JAK inhibitor I (JAK-I) (500 nM) either coincident with infection or 4 days preinfection. Consistent with previous results, interdiction of IFN signaling had no profound effect when initiated at the time of infection, whereas JAK-I added 4 days prior to infection resulted in profound VSV susceptibility in this otherwise resistant cell line.

Vaccinia virus B 18R protein attenuates signaling by the type I IFN receptor (Alcami, A., d al. 2000. J. Virol. 74:11230-11239). Similar to the results with VPA and JAK-I, addition of B18R at the time of infection of SW-R did not increase susceptibility. In contrast, a 4-day preincubation with B18R resulted in profound susceptibility. Consistent with the hypothesis that basal ISG expression correlates with phenotype, 4-day exposure of SW-R to B18R reduced basal ISG mRNA expression levels, including MxA, ISG-15, OAS-1, and ISG-56, even below those of SW-S (FIG. 12A). B18R acts as a decoy IFN receptor, thereby reducing IFN responses. This suggests that the SW-R cells may indeed release small amounts of IFN; a low basal level of IFN release has been proposed as a general property of other cell types (Taniguchi, T., et al. 2001. Mol. Cell. Biol. 2:378-386). Although evidence of IFN secretion was not found in the bioassay described above, culture medium harvested after a 3-day incubation with the SW-R cells did increase MxA expression 180-fold in human astrocytes compared with unconditioned control medium (FIG. 12B). A similar induction was not seen after a 1-day incubation. These data are consistent with the view that SW-R cells may secrete a low level of IFN in the absence of virus infection.

### Example 15

### VSV-Resistant Cells Show Reduced Level of IFN-β Induction

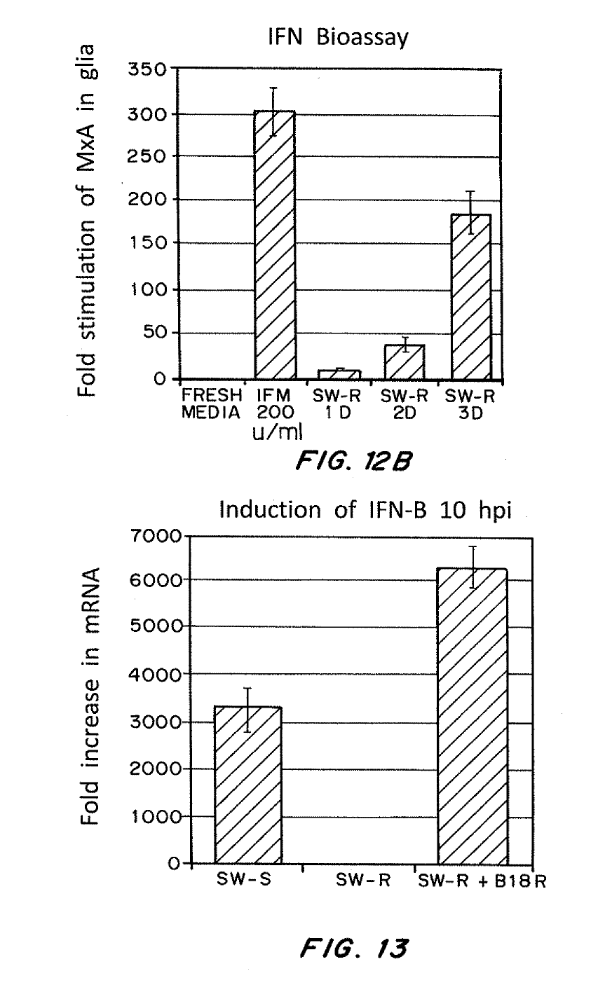

As described above, when the induction of IFN-β by virus was initially studied in VSV-resistant SW982 cells, basal mRNA levels were found to be comparable to those observed in susceptible cells, and only a moderate induction of IFN-β mRNA postinfection was observed. Now having tools in hand to reverse the phenotype of SW-R, the issue of IFN response to infection was revisited. SW-S, SW-R, and SW-R made VSV susceptible by a 4-day pretreatment with B18R were examined for IFN-β mRNA 10 h after infection (or mock infection) with VSV (MOI, 1). Interestingly, a robust postinfection upregulation of IFN-β expression was found in VSV-sensitive cells: specifically, a 3.300-fold upregulation in SW-S and a 6.300-fold upregulation in SW-R pretreated with BIER was found (FIG. 13). However, in contrast, only a modest 23-fold increase of IFN-β was observed in VSV-resistant SW-R not treated with B18R, suggesting that early abrogation of infection in highly resistant cells, mediated by constitutively expressed ISG effectors of innate immunity, precludes stimulation of the signaling cascades that would otherwise lead to upregulation of IFN-β mRNA. These results support the view that it is upregulated basal ISG levels, and not an exaggerated de novo response to infection, that enhances VSV resistance in SW982.

### Example 16

### JAK Inhibitor Enhances VSV Infection of VSV-Resistant Liposarcoma and Bladder Carcinoma

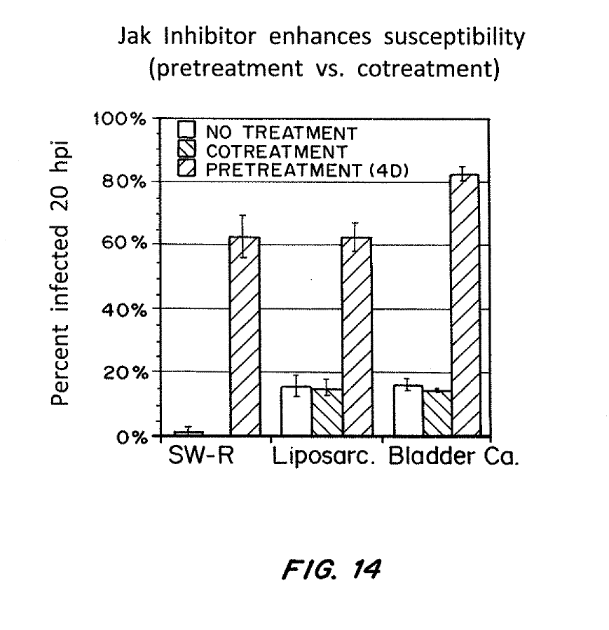

To investigate whether the mechanism of attenuation of SW-R resistance generalized to any other mesodermal tumors, the effect of JAK-I on relatively VSV-resistant liposarcoma was studied. To ask if the mechanism is restricted to sarcomas or would generalize to nonmesodermal carcinomas, a relatively VSV-resistant bladder carcinoma, T24, was also tested. Infectivity 20 hpi with VSV-rp30a was measured, employing SW-R as a control. Similar to SW-R, both the liposarcoma and the bladder carcinoma were rendered more susceptible to VSV-rp30a by JAK-I pretreatment (4 days) but not by cotreatment (FIG. 14). Pretreatment increased the percentage of cells infected 4.0-fold for the liposarcoma (P=−0.001) and 5.1-fold for the bladder carcinoma (P=0.0001), whereas cotreatment had no significant effect.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A pharmaceutical dosage unit composition comprising
an oncolytic vesicular stomatitis virus (VSV) comprising a ratio of replication of at least 1:250 for normal cells:tumor cells and at least two mechanisms of attenuation wherein one of the mechanisms of attenuation is a gene shift comprising movement of the G-gene to the fifth position of the VSV genome
in an amount effective to reduce tumor burden in a patient in need thereof, and a pharmaceutically acceptable carrier.

2. The composition of claim 1 wherein the gene shift comprises insertion of a transgene or reporter gene.

3. The composition of claim 2 wherein the insertion is upstream of the first position of the VSV genome.

4. The composition of claim 3 wherein the transgene is selected from the group consisting of GFP, RFP, and a therapeutic protein.

5. The composition of claim 4 wherein the therapeutic protein in an interferon.

6. The composition of claim 1 wherein one of the mechanisms of attenuation is isolation of the VSV from recombinant VSV derived from DNA plasmid.

7. The composition of claim 1 wherein the VSV virus further comprises a mechanism of attenuation selected from the group consisting of M protein mutations and deletions, G protein truncations and deletions, P or L protein substitutions, gene switching, and genome rearrangements.

8. The composition of claim 1 wherein the VSV virus is VSV-1p-GFP or VSV-1p-RFP.

9. The composition of claim 1 formulated for intranasal, systemic, or local delivery.

10. A method for treating a tumor comprising administering to a subject in need thereof, a pharmaceutical dosage unit composition comprising an oncolytic vesicular stomatitis virus (VSV) comprising a ratio of replication of at least 1:250 for normal cells:tumor cells and at least two mechanisms of attenuation wherein one of the mechanisms of attenuation is a gene shift comprising movement of the G-gene to the fifth position of the VSV genome
in an amount effective to reduce tumor burden.

11. The method of claim 10 wherein the gene shift comprises insertion of a transgene or reporter gene.

12. The method of claim 11 wherein the insertion is upstream of the first position of the VSV genome.

13. The method of claim 12 wherein the transgene is selected from the group consisting of GFP, RFP, and a therapeutic protein.

14. The method of claim 13 wherein the therapeutic protein in an interferon.

15. The method of claim 10 wherein one of the mechanisms of attenuation is isolation of the VSV from recombinant VSV derived from DNA plasmid.

16. The method of claim 10 wherein the VSV virus further comprises a mechanism of attenuation selected from the group consisting of M protein mutations and deletions, G protein truncations and deletions, P or L protein substitutions, gene switching, and genome rearrangements.

17. The method of claim 10 wherein the VSV virus is VSV-1p-GFP or VSV-1p-RFP.

18. The method of claim 10 wherein the tumor comprises cancer selected from the group consisting of bone, bladder, brain, breast, cervical, colo-rectal, esophageal, kidney, liver, lung, nasopharangeal, pancreatic, prostate, skin, stomach, and uterine.

19. The method of claim 10 wherein the tumor comprises a vascular cancer such as multiple myeloma, an adenocarcinomas or a sarcoma.

20. The method of claim 10 wherein the tumor is a brain tumor selected from the group consisting of glioblastomas, oligodendrogliomas, meningiomas, supratentorial ependymonas, pineal region tumors, medulloblastomas, cerebellar astrocytomas, infratentorial ependymonas, brainstem gliomas, schwannomas, pituitary tumors, craniopharyngiomas, optic gliomas, and astrocytomas.

21. The method of claim 10 wherein the pharmaceutical dosage unit is administered by intranasal, local, or systemic delivery.

22. The method of claim 10, wherein the viral dosage unit is administered in combination with a second therapeutic agent selected from the group consisting of immunosuppressants, anticancer agents, and therapeutic proteins.
